(19) United States
(12) Patent Application Publication
(10) Pub. No.: US 2025/0100980 A1
(43) Pub. Date: Mar. 27, 2025

(54) NOVEL LEUKOTRIENE B4 RECEPTOR INHIBITORS AND USE THEREOF

(71) Applicants: EWHA UNIVERSITY - INDUSTRY COLLABORATION FOUNDATION, Seoul (KR); DONGGUK UNIVERSITY INDUSTRY-ACADEMIC COOPERATION FOUNDATION, Seoul (KR)

(72) Inventors: Hyun Ae WOO, Seoul (KR); Hea-Young PARK, Seoul (KR); Jihye YOO, Cheongju-si (KR); Darong KIM, Daegu (KR); Joohye KIM, Gwangmyeong-si (KR); Chang-Hoon LEE, Goyang-si (KR); Hyun Ji KIM, Seoul (KR)

(73) Assignees: EWHA UNIVERSITY - INDUSTRY COLLABORATION FOUNDATION, Seoul (KR); DONGGUK UNIVERSITY INDUSTRY-ACADEMIC COOPERATION FOUNDATION, Seoul (KR)

(21) Appl. No.: 18/723,672

(22) PCT Filed: Nov. 25, 2022
(86) PCT No.: PCT/KR2022/018830

§ 371 Date: Jun. 24, 2024

(30) Foreign Application Priority Data
Dec. 24, 2021 (KR) 10-2021-0187391

(51) Int. Cl. C07D 263/58; A61K 31/423; A61K 31/424; C07C 335/18; C07D 213/75; C07D 498/04

(52) U.S. Cl. CPC C07D 263/58; A61K 31/423; A61K 31/424; C07C 335/18; C07D 213/75; C07D 498/04

(57) ABSTRACT

The present invention relates to N-phenylbenzoxazolo-2-amine and N-phenyloxazolopyridin-2-amine derivatives as novel leukotriene B4 receptor inhibitors, and use thereof.

### TECHNICAL FIELD

The present invention relates to N-phenylbenzoxazolo-2-amine and N-phenyloxazolopyridin-2-amine derivatives as novel leukotriene B4 receptor inhibitors, and use thereof.

### BACKGROUND ART

As leukotriene (LT), synthesized from arachidonic add by 5-lipoxygenase (5-LO), including peptides LTs (LTC4, LTD4, and LTE4) where leukotriene B4 (LTB4) and cysteine are bound, are known as important bioactive substances that mediate acute and chronic inflammation. LTC4, LTD4, and LTE4 induce strong and sustainable contraction of airway or intestinal smooth muscle and secretion of airway mucus, and act as important mediators of allergic bronchial asthma. In addition, various physiological activities such as vascular contraction and relaxation, promotion of neutrophil adhesion, and regulation of macrophage function are also known. Meanwhile, LTB4 exhibits powerful polymorphonuclear leukocyte activation (chemotaxis, aggregation, release of lysosomal enzymes, production of active oxygen, etc.) and plays an important role in inflammation and immune mechanisms, such as the regulation of functions of T-lymphocytes, monocytes, and NK cells. That is, LTB4 induces several inflammatory responses including leukocyte activation, chemotaxis, and degranulation, and the overproduction of LTB4 affects inflammation-related diseases such as bronchial asthma and rheumatoid arthritis.

Two receptors, BLT1 and BLT2, are known for LTB4. BLT1 is widely expressed on inflammatory cells such as leukocytes and is involved in inflammatory processes. BLT2, which has a low affinity for LTB4, is widely expressed in several tissues and is expressed in the spleen, leukocytes, and ovaries at high levels. BLT2 is a downstream component of the oncogene Ras and is known to mediate the transformation of Ras. 5-LO and LTB4 receptors are highly expressed in human pancreatic cancers, but not in normal pancreatic duct tissues. It has been reported that LY293111, which is an antagonist of the LTB4 receptor BLT1, inhibits growth and induces apoptosis of human pancreatic cancer cells, and it has been reported that LY255283, which is an antagonist of BLT2, not only has the activity of inducing the apoptosis of bladder, prostate, pancreas, and breast cancer, but also has the effect of suppressing the metastasis of cancer cells.

### DISCLOSURE

### Technical Problem

The present inventors have made many efforts to discover a novel small molecule compound as an LTB4 inhibitor. As a result, they found that a series of compounds comprising benzoxazole or oxazolopyridine structures were synthesized, and these compounds exhibited an LTB4 inhibitory effect by inhibiting BLT2. Based on this finding, the present invention was completed.

### Technical Solution

One object of the present invention is to provide a series of compounds comprising a benzoxazole or oxazolopyridine structure, or pharmaceutically acceptable salts thereof.

Another object of the present invention is to provide a method of producing the compound comprising a benzoxazole or oxazolopyridine structure, or a pharmaceutically acceptable salt thereof.

Still another object of the present invention is to provide a pharmaceutical composition for the prevention or treatment of diseases related to LTB4 receptor activity, the composition comprising the compound comprising a benzoxazole or oxazolopyridine structure, or a pharmaceutically acceptable salt thereof, as an active ingredient.

Still another object of the present invention is to provide a method of treating diseases related to LTB4 receptor activity, the method comprising a step of administering the pharmaceutical composition to a subject in need thereof.

### Advantageous Effects

Since a series of compounds comprising a benzoxazole or oxazolopyridine structure, newly synthesized according to the present invention, exhibits an LTB4 inhibitory effect by inhibiting BLT2, they can be usefully used in the prevention or treatment of diseases related thereto, such as inflammatory diseases, cardiovascular diseases, and/or cancer diseases.

### BRIEF DESCRIPTION OF THE DRAWING

FIG. 1 shows graphs illustrating the inhibitory activity of compounds according to an embodiment of the present invention on cell migration by LTB4.

FIG. 2 shows graphs illustrating the inhibitory activity of compounds according to an embodiment of the present invention on cell migration and invasion by compound A.

FIG. 3 shows graphs illustrating the concentration-dependent inhibitory effect of the compound of Assay 5 according to an embodiment of the present invention on cell migration by BLT2.

FIG. 4 shows graphs illustrating the concentration-dependent inhibitory effect of the compound of Assay 7 according to an embodiment of the present invention on cell migration by BLT2.

FIG. 5 shows graphs illustrating the concentration-dependent inhibitory effect of the compound of Assay 15 according to an embodiment of the present invention on cell migration by BLT2.

FIG. 6 shows graphs illustrating the concentration-dependent inhibitory effect of the compound of Assay 21 according to an embodiment of the present invention on cell migration by BLT2.

FIG. 7 shows graphs illustrating the inhibitory activity of the compounds of Assays 5, 7, 15, and 21 according to an embodiment of the present invention and LY255283, a known BLT2 agonist, on cell migration and invasion by compound A.

### DETAILED DESCRIPTION OF PREFERRED EMBODIMENTS

Each description and embodiment disclosed in the present invention can also be applied to each other description and embodiment. That is, all combinations of the various elements disclosed in the present invention belong to the scope of the present invention. Additionally, it cannot be presumed that the scope of the present invention is limited by the specific description described below.

Additionally, those skilled in the art can recognize or ascertain many equivalents to specific embodiments of the present invention described herein using only ordinary experiments. Additionally, such equivalents are intended to be included by this invention.

Moreover, throughout the specification of the present invention, when a part is said to “comprise” a certain component, this means that it does not exclude other components but may further include other components unless specifically stated to the contrary.

Hereinafter, the present invention will be described in more detail.

A first aspect of the present invention provides a compound represented by Formula 1 below or a pharmaceutically acceptable salt thereof:

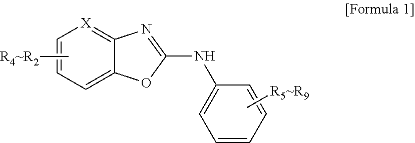

in Formula 1 above,
X is N or CR₁;
R₁ to R₄ are each independently hydrogen, halogen, nitro, C₁₋₄ alkyl, or C₁₋₄ alkoxy; and
R₅ to R₉ are each independently hydrogen, amino, nitro, C₁₋₄ alkoxy, hydroxy-C₁₋₄ alkylcarbonylamino, C₁₋₄ alkylcarbonyloxy-C₁₋₄ alkylcarbonylamino, amino-C₁₋₄ alkylcarbonylamino, or tert-butoxycarbonylamino-C₁₋₄ alkylcarbonylamino.

For example, in Formula 1, R₁ to R₄ may each independently be hydrogen, fluoro, chloro, bromo, nitro, ethyl, tert-butyl, or methoxycydohexylmethyl, but the present invention is not limited thereto.

For example, in Formula 1, R₅ to R₉ are each independently hydrogen, amino, nitro, methoxy, hydroxymethylcarbonylamino, methylcarbonyloxymethylcarbonylamino, tert-butoxycarbonylaminopropylcarbonylamino. tert-butoxycarbonylaminoethylcarbonylamino, or aminopropylcarbonylaminocydohexylmethyl, but the present invention is not limited thereto.

Specifically, the compound may be as follows, but not limited thereto:
1. 5-fluoro-N-(2-nitrophenyl)benzo[d]oxazol-2-amine,
2. 5-fluoro-N-(3-nitrophenyl)benzo[d]oxazol-2-amine,
3. 5-fluoro-N-(2-methoxy-4-nitrophenyl)benzo[d]oxazol-2-amine,
4. 6-fluoro-N-(3-nitrophenyl)benzo[d]oxazol-2-amine,
5. 6-fluoro-N-(4-nitrophenyl)benzo[d]oxazol-2-amine,
6. 6-fluoro-N-(2-methoxy-4-nitrophenyl)benzo[d]oxazol-2-amine,
7. 5-chloro-N-(2-nitrophenyl)benzo[d]oxazol-2-amine,
8. 5-chloro-N-(4-nitrophenyl)benzo[d]oxazol-2-amine,
9. 6-chloro-N-(3-nitrophenyl)benzo[d]oxazol-2-amine,
10. 6-chloro-N-(2-methoxy-4-nitrophenyl)benzo[d]oxazol-2-amine,
11. 5,7-dichloro-N-(2-nitrophenyl)benzo[d]oxazol-2-amine,
12. 5,7-dichloro-N-(3-nitrophenyl)benzo[d]oxazol-2-amine,
13. 5,7-dichloro-N-(4-nitrophenyl)benzo[d]oxazol-2-amine,
14. 5,7-dichloro-N-(2-methoxy-4-nitrophenyl)benzo[d]oxazol-2-amine,
15. 5-chloro-N-(2-methoxy-4-nitrophenyl)-6-nitrobenzo[d]oxazol-2-amine,
16. 7-chloro-N-(2-methoxy-4-nitrophenyl)-5-nitrobenzo[d]oxazol-2-amine,
17. 5,7-dichloro-6-ethyl-N-(2-methoxy-4-nitrophenyl)benzo[d]oxazol-2-amine,
18. 5-tert-butyl-N-(2-methoxy-4-nitrophenyl)benzo[d]oxazol-2-amine,
19. 5-methoxy-N-(2-methoxy-4-nitrophenyl)benzo[d]oxazol-2-amine,
20. 6-bromo-N-(2-methoxy-4-nitrophenyl)oxazolo[4,5-b]pyridin-2-amine,
21. N1-(5-fluorobenzo[d]oxazol-2-yl)benzene-1,2-diamine,
22. N1-(5-fluorobenzo[d]oxazol-2-yl)benzene-1,3-diamine,
23. N1-(5-fluorobenzo[d]oxazol-2-yl)-2-methoxybenzene-1,4-diamine,
24. N1-(6-fluorobenzo[d]oxazol-2-yl)benzene-1,3-diamine,
25. N1-(6-fluorobenzo[d]oxazol-2-yl)benzene-1,4-diamine,
26. N1-(6-fluorobenzo[d]oxazol-2-yl)-2-methoxybenzene-1,4-diamine,
27. N1-(5-chlorobenzo[d]oxazol-2-yl)benzene-1,2-diamine,
28. N1-(5-chlorobenzo[d]oxazol-2-yl)benzene-1,4-diamine,
29. N1-(6-chlorobenzo[d]oxazol-2-yl)benzene-1,3-diamine,
30. N1-(6-chlorobenzo[d]oxazol-2-yl)-2-methoxybenzene-1,4-diamine,
31. N1-(5,7-dichlorobenzo[d]oxazol-2-yl)benzene-1,2-diamine,
32. N1-(5,7-dichlorobenzo[d]oxazol-2-yl)benzene-1,3-diamine,
33. N1-(5,7-dichlorobenzo[d]oxazol-2-yl)benzene-1,4-diamine,
34. N1-(5,7-dichlorobenzo[d]oxazol-2-yl)-2-methoxybenzene-1,4-diamine,
35. N1-(5-tert-butylbenzo[d]oxazol-2-yl)-2-methoxybenzene-1,4-diamine,
36. 2-methoxy-N1-(5-methoxybenzo[d]oxazol-2-yl)benzene-1,4-diamine,
37. 2-(6-chlorobenzo[d]oxazol-2-ylamino)-5-nitrophenol,
38. 2-(2-(5-fluorobenzo[d]oxazol-2-ylamino)phenylamino)-2-oxoethyl acetate,
39. 2-(3-(5-fluorobenzo[d]oxazol-2-ylamino)phenylamino)-2-oxoethyl acetate,
40. 2-(4-(5-fluorobenzo[d]oxazol-2-ylamino)-3-methoxyphenylamino)-2-oxoethyl
acetate,
41. 2-(3-(6-fluorobenzo[d]oxazol-2-ylamino)phenylamino)-2-oxoethyl acetate,
42. 2-(4-(6-fluorobenzo[d]oxazol-2-ylamino)phenylamino)-2-oxoethyl acetate,
43. 2-(4-(6-fluorobenzo[d]oxazol-2-ylamino)-3-methoxyphenylamino)-2-oxoethyl
acetate,
44. 2-(2-(5-chlorobenzo[d]oxazol-2-ylamino)phenylamino)-2-oxoethyl acetate,
45. 2-(4-(6-chlorobenzo[d]oxazol-2-ylamino)phenylamino)-2-oxoethyl acetate,
46. 2-(3-(6-chlorobenzo[d]oxazol-2-ylamino)phenylamino)-2-oxoethyl acetate,
47. 2-(2-(5,7-dichlorobenzo[d]oxazol-2-ylamino)phenylamino)-2-oxoethyl acetate,
48. 2-(3-(5,7-dichlorobenzo[d]oxazol-2-ylamino)phenylamino)-2-oxoethyl acetate,
49. 2-(4-(5,7-dichlorobenzo[d]oxazol-2-ylamino)phenylamino)-2-oxoethyl acetate,
50. 2-(4-(5,7-dichlorobenzo[d]oxazol-2-ylamino)-3-methoxyphenylamino)-2-oxoethyl acetate,
51. N-(2-(5-fluorobenzo[d]oxazol-2-ylamino)phenyl)-2-hydroxyacetamide,
52. N-(3-(5-fluorobenzo[d]oxazol-2-ylamino)phenyl)-2-hydroxyacetamide,
53. N-(4-(5-fluorobenzo[d]oxazol-2-ylamino)-3-methoxyphenyl)-2-hydroxyacetamide,
54. N-(3-(6-fluorobenzo[d]oxazol-2-ylamino)phenyl)-2-hydroxyacetamide,
55. N-(4-(6-fluorobenzo[d]oxazol-2-ylamino)phenyl)-2-hydroxyacetamide,
56. N-(4-(6-fluorobenzo[d]oxazol-2-ylamino)-3-methoxyphenyl)-2-hydroxyacetamide,
57. N-(2-(5-chlorobenzo[d]oxazol-2-ylamino)phenyl)-2-hydroxyacetamide,
58. N-(3-(6-chlorobenzo[d]oxazol-2-ylamino)phenyl)-2-hydroxyacetamide,
59. N-(2-(5,7-dichlorobenzo[d]oxazol-2-ylamino)phenyl)-2-hydroxyacetamide,
60. N-(3-(5,7-dichlorobenzo[d]oxazol-2-ylamino)phenyl)-2-hydroxyacetamide,
61. N-(4-(5,7-dichlorobenzo[d]oxazol-2-ylamino)phenyl)-2-hydroxyacetamide,
62. N-(4-(5,7-dichlorobenzo[d]oxazol-2-ylamino)-3-methoxyphenyl)-2-hydroxyacetamide,
63. tert-butyl 3-(4-(5-chlorobenzo[d]oxazol-2-ylamino)phenylamino)-3-oxopropylcarbamate,
64. tert-butyl 4-(4-(5-chlorobenzo[d]oxazol-2-ylamino)phenylamino)-4-oxobutylcarbamate,
65. tert-butyl 4-(4-(5-tert-butylbenzo[d]oxazol-2-ylamino)-3-methoxyphenylamino)-4-oxobutylcarbamate,
66. tert-butyl 4-(3-methoxy-4-(5-methoxybenzo[d]oxazol-2-ylamino)phenylamino)-4-oxobutylcarbamate, or
67. 4-amino-N-(3-methoxy-4-(5-methoxybenzo[d]oxazol-2-ylamino)phenyl)butanamide.

The compounds of the present invention may exist in the form of pharmaceutically acceptable salts. As the salts, add (addition) salts formed with pharmaceutically acceptable free acids are useful. As used herein, the term “pharmaceutically acceptable salt” refers to any or all organic or inorganic addition salts of the above compounds, in which the side effects caused by this salt do not reduce the beneficial efficacy of the compound represented by Formula 1 at a concentration having an effective action that is relatively non-toxic and harmless to a patient.

Acid addition salts are prepared by conventional methods, for example, a method in which an add addition salt is prepared by dissolving the compound in an excess of aqueous add and precipitating this salt using a water-miscible organic solvent, such as methanol, ethanol, acetone, or acetonitrile. In particular, equimolar amounts of the compound and an add or alcohol (e.g., glycol monomethyl ether) in water may be heated, and then the mixture may be evaporated to dryness, or the precipitated salt may be suction-filtered.

In this case, as the freed acid, an organic add and an inorganic acid may be used. As the inorganic add, hydrochloric add, phosphoric add, sulfuric add, nitric add, tartaric add, or the like may be used. As the organic add, methanesulfonic add, p-toluenesulfonic add, acetic add, trifluoroacetic add, maleic add, succinic add, oxalic add, benzoic acid, tartaric add, fumaric acid, manderic add, propionic add, citric add, lactic add, glycolic add, gluconic add, galacturonic acid, glutamic add, glutaric add, glucuronic add, aspartic add, ascorbic add, carboxylic acid, vanillic add, hydroiodic add, or the like may be used, but the present invention is not limited thereto.

In addition, a pharmaceutically acceptable metal salt may be prepared using a base. The alkali metal salt or alkali earth metal salt is obtained, for example, by dissolving the compound in an excess of an alkali metal hydroxide solution or an alkali earth metal hydroxide solution, filtering an insoluble compound salt, and then evaporating and drying a filtrate. In this case, it is pharmaceutically suitable to prepare a metal salt, especially a sodium, potassium, or calcium salt, but is not limited thereto. In addition, the corresponding silver salt may be obtained by reacting an alkali metal or an alkali earth metal salt with an appropriate silver salt (e.g., silver nitrate).

The pharmaceutically acceptable salts of the compounds of the present invention comprise salts of acidic or basic groups that may be present in the compound of Formula 1, unless otherwise indicated. For example, the pharmaceutically acceptable salts may comprise sodium, calcium, and potassium salts of hydroxyl groups, and other pharmaceutically acceptable salts of amino groups may comprise hydrobromide, sulfate, hydrogen sulfate, phosphate, hydrogen phosphate, dihydrogen phosphate, acetate, succinate, citrate, tartrate, lactate, mandelate, methanesulfonate (mesylate), p-toluenesulfonate (tosylate) salts, and the like, and may be obtained through salt preparation methods known in the art.

Salts of N-phenylbenzoxazolo-2-amine and N-phenyloxazolopyridin-2-amine derivative compounds of the present invention may comprise pharmaceutically acceptable salts, such as salts of N-phenylbenzoxazolo-2-amine and N-phenyloxazolopyridin-2-amine derivatives that exhibit pharmacological activity equivalent to that of N-phenylbenzoxazolo-2-amine and N-phenyloxazolopyridin-2-amine derivative compounds. These salts may be used without limitation.

In addition, the compound represented by Formula 1 according to the present invention comprises, without limitation, not only pharmaceutically acceptable salts thereof, but also solvates such as possible hydrates that can be prepared therefrom, and all possible stereoisomers. Solvates and stereoisomers of the compound represented by Formula 1 may be prepared from the compound represented by Formula 1 using methods known in the art.

Furthermore, the compound represented by Formula 1 according to the present invention may be prepared in a crystalline form or an amorphous form, and when this compound is prepared in a crystalline form, it may be arbitrarily hydrated or solvated. In the present invention, not only stoichiometric hydrates of the compound represented by Formula 1, but also compounds containing various amounts of water may be comprised. The solvates of the compound represented by Formula 1 according to the present invention comprise both stoichiometric solvates and non-stoichiometric solvates.

A second aspect of the present invention provides a method of producing the compound represented by Formula 1 or a pharmaceutically acceptable salt thereof, the method comprising a 1 step of preparing a compound represented by Formula 3 by the cyclization reaction of a hydroxyphenyl-thiourea derivative compound represented by Formula 2 below:

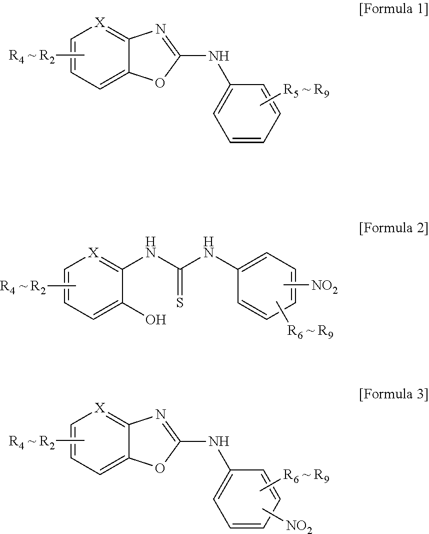

in Formula 1 above,
X is N or CR₁;
R₁ to R₄ are each independently hydrogen, halogen, nitro, C₁₋₄ alkyl, or C₁₋₄ alkoxy; and
R₅ to R₉ are each independently hydrogen, amino, nitro, C₁₋₄ alkoxy, hydroxy-C₁₋₄ alkylcarbonylamino, C₁₋₄ alkylcarbonyloxy-C₁₋₄ alkylcarbonylamino, amino-C₁₋₄ alkylcarbonylamino, or tert-butoxycarbonylamino-C₁₋₄ alkylcarbonylamino.

As used herein, the term “pharmaceutically acceptable salt” is as described above.

For example, the reaction in the 1 step may be performed by reacting with an excess amount of potassium peroxide in an organic solvent, but the present invention is not limited thereto. Specifically, this reaction may be performed by strongly stirring the reaction mixture solution at room temperature for 10 to 20 hours, but the present invention is not limited thereto.

For example, the hydroxyphenyl-thiourea derivative compound represented by Formula 2 may be prepared by the 1′ step of reacting a 2-aminopyridin-3-ol or 2-aminophenol derivative compound represented by Formula 4 and a nitrophenyl isothiocyanate derivative compound represented by Formula 5, but the present invention is not limited thereto:

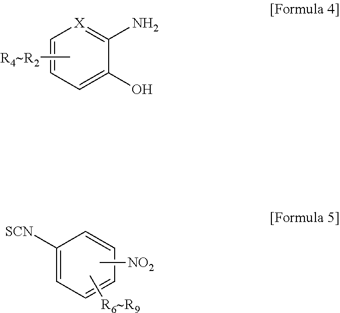

For example, in the 1′ step, the reaction may be performed by mixing reactants, the compound of Formula 4 and the compound of Formula 5, in an equivalent ratio of 1:(0.8 to 1.2) and dissolving these compounds in a lower alcohol, such as methanol. This reaction may be performed while stirring for 18 to 36 hours, but the present invention is not limited thereto.

For example, the production method of the present invention may further comprise a 2 step of reducing the product obtained from the 1 step to convert the nitro group substituted on the phenyl group into an amine group.

For example, the reaction in the 2 step may be performed by hydrogen substitution in the presence of a Pd/C catalyst using a lower alcohol, such as methanol, as a solvent, but the present invention is not limited thereto. For example, this reaction may be performed while stirring at room temperature for 10 to 60 minutes, but the present invention is not limited thereto.

For example, when R₆ is alkoxy, the production method of the present invention may further comprise a 1-1 step of converting the alkoxy group into a hydroxy group through a dealkylation reaction of the product obtained in the 1 step.

For example, the reaction in the 1-1 step may be performed with an excess of boron trifluoride in an organic solvent under nitrogen gas substitution, but the present invention is not limited thereto. For example, this reaction may be performed at a low temperature of −5 to 5° C. while stirring for 18 to 36 hours, but the present invention is not limited thereto.

For example, the production method of the present invention may further comprise a 3-1 step of reacting the product obtained in the 2 step with 2-halo-2-oxy-C₁₋₄ alkyl C₁₋₄ alkanoate to convert the amine group substituted on the phenyl ring into a C₁₋₄ alkylcarbonyloxy-C₁₋₄ alkylcarbonylamino group.

For example, the reaction in the 3-1 step may be performed by adding 2-halo-2-oxy-C₁₋₄ alkyl C₁₋₄ alkanoate and diisopropylethylamine at each equivalent ratio of 0.8 to 1.2 based on the product obtained from the 2 step and reacting them in an organic solvent. This reaction may be performed at room temperature for 12 to 24 hours, but the present invention is not limited thereto.

For example, the production method of the present invention may further comprise a 4-1 step of reacting the product obtained in the 3-1 step under a base to convert the C₁₋₄ alkylcarbonyloxy-C₁₋₄ alkylcarbonylamino group substituted on the phenyl ring into a hydroxy-C₁₋₄ alkylcarbonylamino group.

For example, this reaction may be performed in a lower alcohol solvent, such as ethanol, and may be performed by stirring at room temperature for 10 to 60 hours, but the present invention is not limited thereto.

For example, the production method of the present invention may further comprise a 3-2 step of reacting the product obtained from the 2 step with tert-butoxycarbonylamino-C₁₋₄ alkanoic acid to convert the amine group substituted on the phenyl ring into a tert-butoxycarbonylamino-C₁₋₄alkylcarbonylamino group.

For example, the reaction in the 3-2 step may be performed by adding tert-butoxycarbonylamino-C₁₋₄ alkanoic add, (benzotriazol-1-yloxy)tripyrrolidinophosphonium hexafluorophostate, and diisopropylethylamine. at each equivalent ratio of 0.8 to 1.2 based on the product obtained from the 2 step and reacting them in an organic solvent. This reaction may be performed by stirring at room temperature for 12 to 24 hours, but the present invention is not limited thereto.

For example, the production method of the present invention may further comprise a 4-2 step of reacting the product obtained from the 3-2 step with an add in an organic solvent to remove a tert-butoxycarbonyl group.

For example, the reaction in the 4-2 step may be performed by stirring at room temperature for 1 to 5 hours, but the present invention is not limited thereto.

In the production method of the present invention, in order to increase purity and/or yield, the product after each reaction may be used as is in subsequent reactions, or steps of washing, separating, and/or purifying the product may be additionally performed. The present invention is not limited thereto.

A third aspect of the present invention provides a pharmaceutical composition for preventing or treating diseases related to LTB4 receptor activity, the composition comprising a compound of Formula 1 below or a pharmaceutically acceptable salt thereof, as an active ingredient:

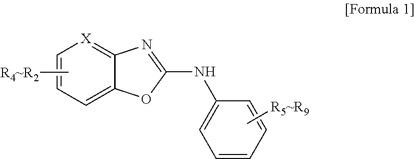

in Formula 1 above,
X is N or CR₁;
R₁ to R₄ are each independently hydrogen, halogen, nitro, C₁₋₄ alkyl, or C₁₋₄ alkoxy; and R₅ to R₉ are each independently hydrogen, amino, nitro, C₁₋₄ alkoxy, hydroxy-C₁₋₄ alkylcarbonylamino, C₁₋₄ alkylcarbonyloxy-C₁₋₄ alkylcarbonylamino, amino-C₁₋₄ alkylcarbonylamino, or tert-butoxycarbonylamino-C₁₋₄ alkylcarbonylamino.

As used herein, the term “pharmaceutically acceptable salt” is as described above.

As used herein, the term “prevention” refers to all actions that inhibit or delay the occurrence, spread, and recurrence of diseases related to LTB4 receptor activity by administering the composition of the present invention, and the term “treatment” refers to all actions by which symptoms of the diseases are improved or beneficially changed by administration of the composition of the present invention.

For example, the pharmaceutical composition of the present invention may exhibit an effect of inhibiting an increase in cell migration, invasion, or both due to activation of BLT2.

In an embodiment of the present invention, it was confirmed that a series of compounds according to the present invention effectively reduces an increase in migration and invasion of cancer cell lines, such as pancreatic cancer cell lines, caused by Compound A, a BLT2 agonist, and that the effect is at the same or similar level to LY255283, a known BLT2 antagonist.

As used herein, the term “leukotriene B4” refers to one of leukotrienes, which are a family of eicosanoid inflammatory mediators produced from leukocytes by the oxidation of arachidonic add and eicosapentaenoic add (EPA), an essential fatty add, by 5-lipooxygenases, and is involved in inflammation. The “leukotriene B4” is synthesized from leukotriene A4 by leukotriene A4 hydrolase. As described above, the leukotriene B4 is produced from leukocytes in response to inflammatory mediators and is bound to tissues by inducing adhesion and activation of leukocytes to endothelium, thereby allowing it to pass the tissues. This the leukotriene B4 is a powerful chemoattractant for neutrophils and can induce the formation of reactive oxygen species and the release of lysosomal enzymes by these cells.

In this case, diseases related to LTB4 receptor activity that can be prevented or treated with the pharmaceutical composition of the present invention may be inflammatory diseases, cardiovascular diseases, or cancer diseases.

For example, the inflammatory disease refers to a general term for diseases whose main lesion is inflammation, and may be an acute or chronic inflammatory disease such as an infectious disease, an allergy, an autoimmune disease, or a metabolic disease. Specifically, the inflammatory diseases may comprise: allergic diseases including allergic asthma, allergic rhinitis, allergic mucositis, urticaria and anaphylaxis; myopathies including systemic sclerosis, dermatomyositis and inclusion body myositis; and arthritis, atopic dermatitis, psoriasis, asthma, multiple sclerosis, ssRNA and dsRNA virus infections, sepsis, polychondritis, scleroderma, eczema, gout, periodontal disease, Behcet syndrome, edema, vasculitis, Kawasaki disease, diabetic retinitis, autoimmune pancreatitis, vasculitis, glomerulonephritis, acute and chronic bronchitis, and influenza infections.

For example, the cardiovascular diseases (CVDs) may comprise coronary heart disease, deep vein thrombosis, vascular calcification, cerebrovascular diseases, peripheral arterial diseases, rheumatic heart disease, and congenital heart disease.

For example, the cancer diseases may comprise colorectal cancer, breast cancer, pancreatic cancer, ovarian cancer, esophageal squamous cell cancer, glioblastomas, malignant melanomas, renal carcinomas, cervical squamous cell carcinomas, hepatocellular carcinomas, and cervical intraepithelial neoplasia.

However, diseases that can be prevented or treated using the pharmaceutical composition of the present invention are not limited thereto, and any disease that can exhibit a preventive or therapeutic effect by inhibiting LTB4 activity may be comprised in the scope of the present invention.

Preferably, the pharmaceutical composition according to the present invention comprises the compound represented by Formula 1 or a pharmaceutically acceptable salt thereof, as an active ingredient. in an amount of 0.1 to 75 wt %, more preferably 1 to 50 wt %, based on the total weight of the composition.

The composition of the present invention may further comprise a pharmaceutically acceptable carrier, diluent or excipient, may be used in various forms such as oral dosage forms such as powders, granules, tablets, capsules, suspensions, emulsions, syrups, aerosols, etc., and injections of sterile injectable solutions for each purpose according to conventional methods, and may be administered orally or through various routes including intravenous, intraperitoneal, subcutaneous, rectal, topical administration, etc. Examples of suitable carriers, excipients or diluents that may be comprised in this composition may include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, mineral oil, and the like. In addition, the composition of the present invention may further comprise a filler, an anti-coagulant, a lubricant, a wetting agent, a fragrant, an emulsifier, a preservative, or the like.

Solid preparations for oral administration include tablets, pills, powders, granules, capsules, etc. These solid preparations are formulated by mixing at least one excipient, such as starch, calcium carbonate, sucrose, lactose, gelatin, etc., with the composition. Further, in addition to simple excipients, lubricants such as magnesium stearate and talc may be used.

Oral liquid preparations may include suspensions, oral solutions, emulsions, syrups, etc. and may contain various excipients, such as a wetting agent, a sweetener, a flavoring agent, a preservative, etc. in addition to commonly used simple diluents such as water and liquid paraffin,

Preparations for parenteral administration include sterilized aqueous solutions, non-aqueous solutions, suspensions, emulsions, lyophilized preparations, and suppositories. As the non-aqueous solvent and suspension, propylene glycol, polyethylene glycol, plant oil such as olive oil, and injectable ester such as ethyl oleate may be used. As the basis of the suppository, Witepsol, Macrogol, Twin61. cacao oil, laurin oil, glycerogeratin, etc. may be used. Meanwhile, injectables may contain conventional additives such as solubilizers, isotonic agents, suspending agents, emulsifiers, stabilizers, preservatives, etc.

In this case, the composition of the present invention is administered in a pharmaceutically effective amount. As used herein, the term “pharmaceutically effective amount” refers to an amount sufficient to treat a disease at a reasonable benefit/risk ratio applicable to medical treatment and not cause side effects, and the level of the effective amount may be determined depending on patient's health status, type of disease, severity, activity of drug, sensitivity to drug, administration method, administration time, administration rout, excretion rate, treatment period, factors including drugs used in combination or concurrently, and other factors well known in the medical fields. The composition of the present invention may be administered as a subject therapeutic agent or administered in combination with other therapeutic agents, may be administered sequentially or simultaneously with a conventional therapeutic agent, and may be administered single or multiple. It is important to administer an amount capable of obtaining the maximum effect in a minimum amount without side effects in consideration of all the above factors, which may be easily determined by those skilled in the art.

For example, since the administration amount may increase or decrease depending on administration route, disease severity, sex, weight, age, and the like, the administration amount does not limit the scope of the present invention in any way.

Specifically, the effective amount of the compound in the composition of the present invention may vary depending on the patient's age, gender, and weight, and, generally, the compound may be administered in an amount of 1 to 100 mg per kg of body weight, preferably 5 to 60 mg per kg of body weight, daily or every other day, or 1 to 3 times per day. However, since the effective amount of the compound may increase or decrease depending on administration route, severity of disease, gender, weight, age, etc., the administration amount does not limit the scope of the present invention in any way.

A fourth aspect of the present invention provides a method of treating diseases related to LTB4 receptor activity, the method comprising a step of administering the pharmaceutical composition of the third aspect to a subject in need thereof.

As used herein, the terms “pharmaceutical composition of the third aspect” and “LTB4 receptor activity-related disease” are as described above.

As used herein, the term “subject” refers to any animal including monkeys, cows, horses, sheep, pigs, chickens, turkeys, quails, cats, dogs, mice, rats, rabbits, and guinea pigs in addition to humans, which have developed or may develop diseases related to LTB4 receptor activity. These diseases may be effectively prevented or treated by administering the pharmaceutical composition of the present invention to the subject. The pharmaceutical composition of the present invention may be administered in combination with an existing therapeutic agent.

As used herein, the term “administration” refers to providing a predetermined substance to a patient by any suitable method, and the administration route of the composition of the present invention may be administered through any general route as long as it can reach target tissues. The composition may be administered through intraperitoneal administration, intravenous administration, intramuscular administration, subcutaneous administration, intradermal administration, oral administration, topical administration, intranasal administration, intrapulmonary administration, or rectal administration, but the present invention is not limited thereto. Meanwhile, the pharmaceutical composition of the present invention may be administered by any device capable of moving an active substance to a target cell. Preferred administrations and formulations include intravenous injection drugs, subcutaneous injection drugs, intradermal injection drugs, intramuscular injection drugs, and dropwise injection drugs. The injection drugs may be prepared using an aqueous solvent such as a physiological saline solution or a Ringer's solution, or a non-aqueous solvent such as plant oil, higher fatty add ester (for example, oleic add ethyl) or alcohol (for example, ethanol, benzyl alcohol, propylene glycol, or glycerin), and may include a pharmaceutical carrier such as a stabilizing agent for preventing denature (for example, ascorbic add, sodium hydrogen sulfite, sodium pyrosulfite, BHA, tocopherol, or EDTA), an emulsifying agent, a buffering agent for pH control, or a preservative for inhibiting the growth of microorganisms (for example, phenylmercury nitrate, thimerosal, benzalkonium chloride, phenol, cresol, or benzyl alcohol).

As used herein, the term “therapeutically effective amount” used in combination with the active ingredient in the present invention refers to the amount of N-phenylbenzoxazolo-2-amine and N-phenyloxazolopyridin-2-amine derivative compounds, or pharmaceutically acceptable salts thereof, which are effective in preventing or treating target diseases.

The pharmaceutical composition of the present invention may further comprise known drugs used for the prevention or treatment of each known disease, as active ingredients, in addition to N-phenylbenzoxazolo-2-amine and N-phenyloxazolopyridin-2-amine derivative compounds, or pharmaceutically acceptable salts thereof, depending on the kind of diseases to be prevent or treat. For example, when used for prevention or treatment of cancer diseases, the pharmaceutical composition may further comprise known drugs as active ingredients, in addition to N-phenylbenzoxazolo-2-amine and N-phenyloxazolopyridin-2-amine derivative compounds, or pharmaceutically acceptable salts thereof, and may be used in combination with other known treatments for the treatment of these diseases.

### MODE FOR CARRYING OUT THE INVENTION

Hereinafter, the configuration and effects of the present invention will be described in more detail with reference to Examples. However, these Examples are only illustrative of the present invention, and the scope of the present invention is not limited to these Examples.

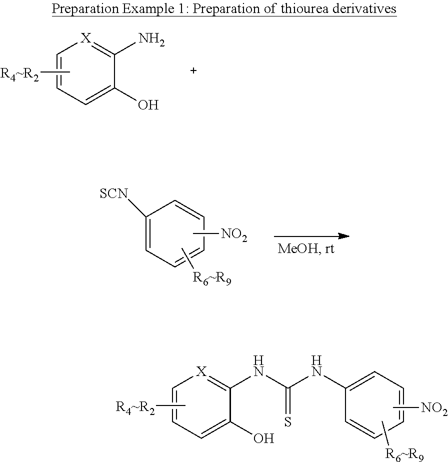

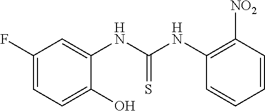 [SMILES: O=[N+]([O-])c1ccccc1NC(=S)Nc1cc(F)ccc1O]

2-amino-4-fluorophenol was mixed with 2-nitrophenyl isothiocyanate in the same equivalent amount, and the mixture was completely dissolved by adding methanol and then reacted with stirring for 24 hours. After confirming the completion of the reaction using thin layer chromatography, the solvent was removed through reduced pressure evaporation to obtain a product.
Greenish brown solid (53.8%);
m.p. 163˜165° C.;

¹H NMR (400 MHz, DMSO-d₆) δ 10.28 (s, 1H), 10.03 (s, 1H), 9.78 (s, 1H), 8.01 (dd, J=8.2, 1.4 Hz, 2H), 7.79 (dd, J=8.0, 1.2 Hz, 1H), 7.72 (ddd, J=8.0, 7.3, 1.5 Hz, 1H), 7.45 (td, J=7.8, 1.5 Hz, 1H), 6.90-6.82 (m, 2H);

HR-FABMS Calcd for C₁₃H₁₀FN₃O₃S (M+H)⁺: 308.0500, Found: 308.0503.

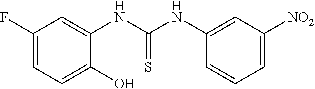 [SMILES: O=[N+]([O-])c1cccc(NC(=S)Nc2cc(F)ccc2O)c1]

The title compound was obtained in a similar manner to Preparation Example 1-1, except that 3-nitrophenyl isothiocyanate was used instead of 2-nitrophenyl isothiocyanate.
Greenish brown solid (49.1%);
m.p. 142˜144° C.;

¹H NMR (400 MHz, DMSO-d₆) δ 10.51 (s, 1H), 10.00 (s, 1H), 9.47 (s, 1H), 8.68 (t, J=2.2 Hz, 1H), 7.99-7.91 (m, 3H), 7.62 (t, J=8.2 Hz, 1H), 6.90-6.82 (m, 2H);

HR-FABMS Calcd for C₁₃H₁₀FN₃O₃S (M+H)⁺: 308.0500, Found: 308.0502.

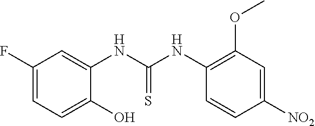 [SMILES: COc1cc([N+](=O)[O-])ccc1NC(=S)Nc1cc(F)ccc1O]

The title compound was obtained in a similar manner to Preparation Example 1-1, except that 4-nitro-2-methoxyphenyl-isothiocyanate was used instead of 2-nitrophenyl isothiocyanate.
White solid (99.3%);
m.p. 143˜145° C.;

¹H NMR (400 MHz, DMSO-d₆) δ10.09 (s, 1H), 10.01 (s, 1H), 9.97 (s, 1H), 8.67 (d, J=9.2 Hz, 1H), 7.93-7.84 (m, 3H), 6.89-6.86 (m, 2H), 4.01 (s, 3H);

HR-FABMS Calcd for C₁₄H₁₃FN₃O₄S (M+H)⁺: 338.0605, Found: 338.0606.

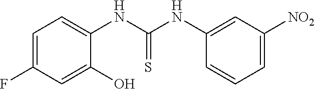 [SMILES: O=[N+]([O-])c1cccc(NC(=S)Nc2ccc(F)cc2O)c1]

The title compound was obtained in a similar manner to Preparation Example 1-1, except that 3-nitrophenyl isothiocyanate was used instead of 2-nitrophenyl isothiocyanate, and 2-amino-5-fluorophenol was used instead of 2-amino-4-fluorophenol.
Brown solid (86.1%);
m.p. 171˜173° C.;

¹H NMR (400 MHz, DMSO-d₆) δ 10.34 (s, 1H), 10.12 (s, 1H), 9.37 (s, 1H), 8.65 (t, J=2.0 Hz, 1H), 7.95 (dq, J=8.0, 1.2 Hz, 1H), 7.91 (dq, J=8.0, 1.1 Hz, 1H), 7.62-7.55 (m, 2H), 6.69 (dd, J=10.6, 3.0 Hz, 1H), 6.64 (td, J=8.6, 2.8 Hz, 1H);

HR-FABMS Calcd for C₁₃H₁₀FN₃O₃S (M+H)⁺: 308.0500, Found: 308.0503.

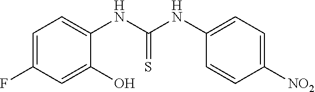 [SMILES: O=[N+]([O-])c1ccc(NC(=S)Nc2ccc(F)cc2O)cc1]

The title compound was obtained in a similar manner to Preparation Example 1-1, except that 4-nitrophenyl isothiocyanate was used instead of 2-nitrophenyl isothiocyanate, and 2-amino-5-fluorophenol was used instead of 2-amino-4-fluorophenol.
Brown solid (70.1%);
m.p. 148˜150° C.;

¹H NMR (400 MHz, DMSO-d₆) δ 10.37 (s, 2H), 9.47 (s, 1H), 8.20 (dt, J=9.2, 2.7 Hz, 2H), 7.93 (dt, J=9.2, 2.6 Hz, 2H), 7.60 (t, J=7.2 Hz, 1H), 6.70-6.61 (m, 2H);

HR-FABMS Calcd for C₁₃H₁₀FN₃O₃S (M+H)⁺: 308.0500, Found: 308.0502.

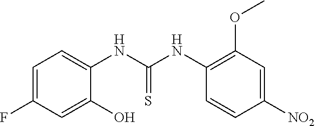 [SMILES: COc1cc([N+](=O)[O-])ccc1NC(=S)Nc1ccc(F)cc1O]

The title compound was obtained in a similar manner to Preparation Example 1-1, except that 4-nitro-2-methoxyphenylisothiocyanate was used instead of 2-nitrophenyl isothiocyanate, and 2-amino-5-fluorophenol was used instead of 2-amino-4-fluorophenol.
Yellow solid (87.7%);
m.p. 141˜143° C.;

¹H NMR (400 MHz, DMSO-d₆) δ 10.35 (s, 1H), 9.97 (s, 1H), 9.69 (s, 1H), 8.81 (d, J=8.8 Hz, 1H), 7.89 (dd, J=9.0, 2.6 Hz, 1H), 7.83 (d, J=2.8 Hz, 1H), 7.60 (t, J=7.2 Hz, 1H), 6.70 (dd, J=10.4, 2.8 Hz, 1H), 6.65 (td, J=8.9, 2.9 Hz, 1H), 4.00 (s, 3H);

HR-FABMS Calcd for C₁₄H₁₂FN₃O₄S (M+H)⁺: 338.0605, Found: 338.0609.

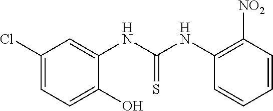 [SMILES: O=[N+]([O-])c1ccccc1NC(=S)Nc1cc(Cl)ccc1O]

The title compound was obtained in a similar manner to Preparation Example 1-1, except that 2-amino-4-chlorophenol was used instead of 2-amino-4-fluorophenol.
Yellow solid (63.7%);
m.p. 157˜158° C.;

¹H NMR (400 MHz, DMSO-d₆) δ 10.31 (s, 1H), 10.23 (s, 1H), 9.78 (s, 1H), 8.09 (d, J=2.4 Hz, 1H), 8.01 (dd, J=8.4, 1.2 Hz, 1H), 7.79 (dd, J=8.0, 1.2 Hz, 1H), 7.71 (td, J=7.6, 1.5 Hz, 1H), 7.44 (td, J=7.8, 1.2 Hz, 1H), 7.06 (dd, J=8.6, 2.6 Hz, 1H), 6.91 (d, J=8.8 Hz, 1H);

HR-FABMS Calcd for C₁₃H₁₀CIN₃O₃S (M+H)⁺: 324.0204, Found: 324.0206.

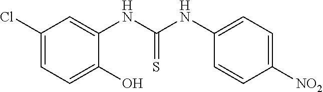 [SMILES: O=[N+]([O-])c1ccc(NC(=S)Nc2cc(Cl)ccc2O)cc1]

The title compound was obtained in a similar manner to Preparation Example 1-1, except that 4-nitrophenyl isothiocyanate was used instead of 2-nitrophenyl isothiocyanate, and 2-amino-4-chlorophenol was used instead of 2-amino-4-fluorophenol.
Yellow solid (73.4%;
m.p. 143˜145° C.;

¹H NMR (400 MHz, DMSO-d₆) 10.67 (s, 1H), 10.31 (s, 1H), 9.59 (s, 1H), 8.22 (dt, J=9.2, 2.6 Hz, 2H), 8.03 (d, J=2.8 Hz, 1H), 7.95 (dt, J=9.6, 2.6 Hz, 2H), 7.07 (dd, J=8.6, 2.6 Hz, 1H), 6.91 (d, J=8.4 Hz, 1H);

HR-FABMS Calcd for C₁₃H₁₀CIN₃O₃S (M+H)⁺: 324.0204, Found: 324.0206.

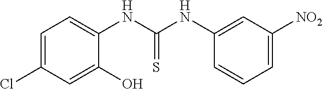 [SMILES: O=[N+]([O-])c1cccc(NC(=S)Nc2ccc(Cl)cc2O)c1]

The title compound was obtained in a similar manner to Preparation Example 1-1, except that 3-nitrophenyl isothiocyanate was used instead of 2-nitrophenyl isothiocyanate, and 2-amino-5-chlorophenol was used instead of 2-amino-4-fluorophenol.
Brown solid (67.0%);
m.p. 164˜165° C.;

¹H NMR (400 MHz, DMSO-d₆) δ 10.42 (s, 1H), 10.27 (s, 1H), 9.44 (s, 1H), 8.66 (t, J=2.2 Hz, 1H), 7.96 (dq, J=8.0, 1.1 Hz, 1H), 7.92 (dq, J=8.0, 1.1 Hz, 1H), 7.74 (d, J=8.8 Hz, 1H), 7.61 (t, J=8.2 Hz, 1H), 6.92 (d, J=2.4 Hz, 1H), 6.86 (dd, J=8.4, 2.4 Hz, 1H);

HR-FABMS Calcd for C₁₃H₁₀CIN₃O₃S (M+H)⁺: 324.0204, Found: 324.0206.

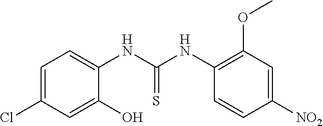 [SMILES: COc1cc([N+](=O)[O-])ccc1NC(=S)Nc1ccc(Cl)cc1O]

The title compound was obtained in a similar manner to Preparation Example 1-1, except that 4-nitro-2-methoxyphenyl-isothiocyanate was used instead of 2-nitrophenyl isothiocyanate and 2-amino-5-chlorophenol was used instead of 2-amino-4-fluorophenol.
Yellowish green solid (80.8%);
m.p. 162˜164° C.;

¹H NMR (400 MHz, DMSO) δ 10.42 (s, 1H), 10.03 (s, 1H), 9.83 (s, 1H), 8.75 (d, J=8.8 Hz, 1H), 7.89 (dd, J=9.0, 2.6 Hz, 1H), 7.83 (d, J=2.8 Hz, 1H), 7.78 (d, J=8.4 Hz, 1H), 6.93 (d, J=2.8 Hz, 1H), 6.86 (dd, J=8.6, 2.6 Hz, 1H), 4.01 (s, 3H);

HR-FABMS Calcd for C₁₄H₁₂CIN₃O₄S (M+H)⁺: 354.0310, Found: 354.0312.

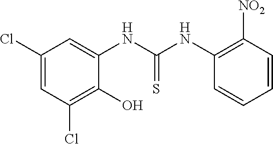 [SMILES: O=[N+]([O-])c1ccccc1NC(=S)Nc1cc(Cl)cc(Cl)c1O]

The title compound was obtained in a similar manner to Preparation Example 1-1, except that 2-amino-4,6-dichlorophenol was used instead of 2-amino-4-fluorophenol.
Greenish brown solid (49.3%);
m.p. 138˜140° C.;

¹H NMR (400 MHz, DMSO-d₆) δ 10.29 (s, 1H), 10.13 (s, 1H), 9.91 (s, 1H), 8.02 (dd, J=8.2, 1.4 Hz, 1H), 7.86 (d, J=2.4 Hz, 1H), 7.79 (dd, J=7.6, 0.8 Hz, 1H), 7.72 (td, J=7.7, 1.4 Hz, 1H), 7.45 (td, J=7.9, 1.4 Hz, 1H), 7.37 (d, J=2.8 Hz, 1H);

HR-FABMS Calcd for C₁₃H₉Cl₂N₃O₃S (M+H)⁺: 356.9742, Found: 356.9744.

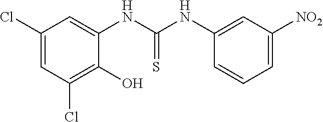 [SMILES: O=[N+]([O-])c1cccc(NC(=S)Nc2cc(Cl)cc(Cl)c2O)c1]

The title compound was obtained in a similar manner to Preparation Example 1-1, except that 3-nitrophenyl isothiocyanate was used instead of 2-nitrophenyl isothiocyanate and 2-amino-5-fluorophenol was used instead of 2-amino-4,6-dichlorophenol.
Brown solid (66.2%);
m.p. 156˜158° C.;

¹H NMR (400 MHz, DMSO-d₆) δ10.53 (s, 1H), 10.11 (s, 1H), 9.57 (s, 1H), 8.65 (t, J=2.2 Hz, 1H), 7.99 (dq, J=8.4, 1.1 Hz, 1H), 7.93 (dq, J=7.6, 1.0 Hz, 1H), 7.75 (d, J=2.4 Hz, 1H), 7.63 (t, J=8.2 Hz, 1H), 7.39 (d, J=2.8 Hz, 1H);

HR-FABMS Calcd for C₁₃H₉Cl₂N₃O₃S (M+H)⁺: 357.9820, Found: 357.9818.

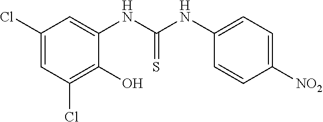 [SMILES: O=[N+]([O-])c1ccc(NC(=S)Nc2cc(Cl)cc(Cl)c2O)cc1]

The title compound was obtained in a similar manner to Preparation Example 1-1, except that 4-nitrophenyl isothiocyanate was used instead of 2-nitrophenyl isothiocyanate and 2-amino-4,6-dichlorophenol was used instead of 2-amino-4-fluorophenol.
Brown solid (68.7%);
m.p. 166˜168° C.;

1H NMR (400 MHz, DMSO-d₆) b 10.82 (s, 1H), 10.13 (s, 1H), 9.71 (s, 1H), 8.22 (d, J=8.8 Hz, 2H), 7.96 (d, J=9.2 Hz, 2H), 7.76 (d, J=2.0 Hz, 1H), 7.39 (d, J=2.0 Hz, 1H);

HR-FABMS Calcd for C₁₃H₉Cl₂N₃O₃S (M+H)⁺: 357.9820, Found: 357.9818.

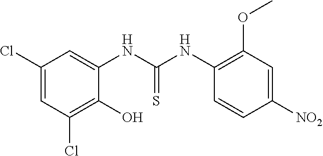 [SMILES: COc1cc([N+](=O)[O-])ccc1NC(=S)Nc1cc(Cl)cc(Cl)c1O]

The title compound was obtained in a similar manner to Preparation Example 1-1, except that 4-nitro-2-methoxyphenyl-isothiocyanate was used instead of 2-nitrophenyl isothiocyanate and 2-amino-4,6-dichlorophenol was used instead of 2-amino-4-fluorophenol.
Brown solid (50.9%);
m.p. 189˜191° C.;

¹H-NMR (DMSO-d_{6,} 400 MHz) δ 10.16 (s, 1H), 10.11 (s, 1H), 9.98 (s, 1H), 8.71 (d, J=9.2 Hz, 1H), 7.90 (dd, J=8.8, 2.4 Hz, 1H), 7.85 (d, J=2.8 Hz, 1H), 7.80 (d, J=2.8 Hz, 1H), 7.39 (d, J=2.8 Hz, 1H), 4.03 (s, 3H);

HR-FABMS Calcd for C₁₄H₁₁Cl₂N₃O₄S (M+H)⁺: 337.9922, Found: 337.9922.

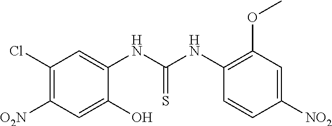 [SMILES: COc1cc([N+](=O)[O-])ccc1NC(=S)Nc1cc(Cl)c([N+](=O)[O-])cc1O]

The title compound was obtained in a similar manner to Preparation Example 1-1, except that 4-nitro-2-methoxyphenyl-isothiocyanate was used instead of 2-nitrophenyl isothiocyanate, and 2-amino-4-chloro-5-nitrophenol was used instead of 2-amino-4-fluorophenol.

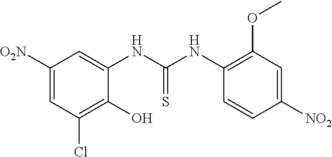 [SMILES: COc1cc([N+](=O)[O-])ccc1NC(=S)Nc1cc([N+](=O)[O-])cc(Cl)c1O]

The title compound was obtained in a similar manner to Preparation Example 1-1, except that 4-nitro-2-methoxyphenyl-isothiocyanate was used instead of 2-nitrophenyl isothiocyanate, and 2-amino-6-chloro-4-nitrophenol was used instead of 2-amino-4-fluorophenol.

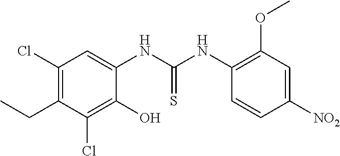 [SMILES: CCc1c(Cl)cc(NC(=S)Nc2ccc([N+](=O)[O-])cc2OC)c(O)c1Cl]

The title compound was obtained in a similar manner to Preparation Example 1-1, except that 4-nitro-2-methoxyphenyl-isothiocyanate was used instead of 2-nitrophenyl isothiocyanate, and 6-amino-2,4-dichloro-3-ethylphenol was used instead of 2-amino-4-fluorophenol.

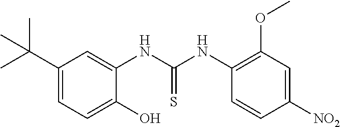 [SMILES: COc1cc([N+](=O)[O-])ccc1NC(=S)Nc1cc(C(C)(C)C)ccc1O]

The title compound was obtained in a similar manner to Preparation Example 1-1, except that 4-nitro-2-methoxyphenyl-isothiocyanate was used instead of 2-nitrophenyl isothiocyanate, and 2-amino-4-tert-butylphenol was used instead of 2-amino-4-fluorophenol.
Yellow solid (87.9%);
m.p. 150˜153° C.;

¹H NMR (400 MHz, DMSO-d₆) δ 10.03 (s, 1H), 9.68 (s, 2H), 8.81 (d, J=8.8 Hz, 1H), 7.88 (dd, J=8.8, 2.4 Hz, 1H), 7.82 (d, J=2.4 Hz, 1H), 7.74 (s, 1H), 7.08 (dd, J=8.8, 2.4 Hz, 1H), 6.84 (d, J=8.4 Hz, 1H), 3.89 (s, 3H), 1.24 (s, 9H);

HR-FABMS Calcd for C₁₈H₂₂N₃O₄S (M+H)⁺: 376.1326, Found: 376.1326.

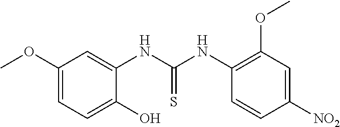 [SMILES: COc1ccc(O)c(NC(=S)Nc2ccc([N+](=O)[O-])cc2OC)c1]

The title compound was obtained in a similar manner to Preparation Example 1-1, except that 4-nitro-2-methoxyphenyl-isothiocyanate was used instead of 2-nitrophenyl isothiocyanate, and 2-amino-4-methoxyphenol was used instead of 2-amino-4-fluorophenol.
Yellow solid (94.0%);
m.p. 144˜147° C.;

¹H NMR (400 MHz, DMSO-d₆) δ 10.01 (s, 1H), 9.83 (s, 1H), 9.46 (s, 1H), 8.72 (d, J=8.8 Hz, 1H), 7.89-7.82 (m, 2H), 7.48 (s, 1H), 6.83 (d, J=8.8 Hz, 1H), 6.66-6.63 (m, 1H), 3.99 (s, 3H), 3.67 (s, 3H);

HR-FABMS Calcd for C₁₅H₁₆N₃O₅S (M+H)⁺: 350.0807, Found: 350.0805.

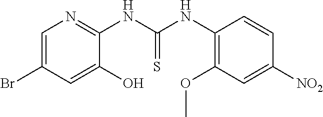 [SMILES: COc1cc([N+](=O)[O-])ccc1NC(=S)Nc1ncc(Br)cc1O]

The title compound was obtained in a similar manner to Preparation Example 1-1, except that 4-nitro-2-methoxyphenyl-isothiocyanate was used instead of 2-nitrophenyl isothiocyanate, and 2-amino-5-bromopyridin-3-ol was used instead of 2-amino-4-fluorophenol.

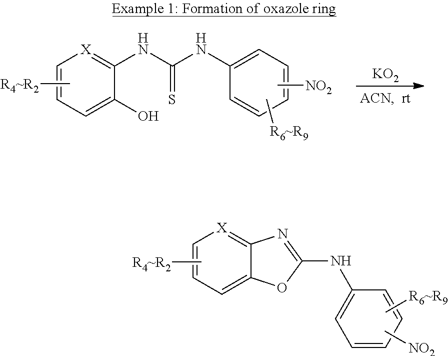

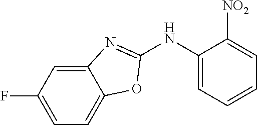 [SMILES: O=[N+]([O-])c1ccccc1Nc1nc2cc(F)ccc2o1]

A mixture of 5 equivalents of potassium peroxide and 5 mL of acetonitrile was added to 1 equivalent of 1-(5-fluoro-2-hydroxyphenyl)-3-(2-nitrophenyl)thiourea prepared according to Preparation Example 1-1 under nitrogen gas substitution, and then reacted with strong stirring at room temperature for 16 hours. Then, the reaction product was diluted with cold distilled water and extracted with dichloromethane. The product was washed with saturated aqueous sodium chloride solution, dried over magnesium sulfate, filtered, and then a solvent was removed by evaporation under reduced pressure to obtain the title compound.
Yellow solid (65.2%);
m.p. 159˜161° C.;

¹H NMR (400 MHz, DMSO) δ 10.73 (s, 1H), 8.23 (s, 1H), 8.11 (d, J=8.4 Hz, 1H), 7.81 (t, J=7.6 Hz, 1H), 7.57 (dd, J=8.4, 4.4 Hz, 1H), 7.36 (t, J=8.2 Hz, 1H), 7.31 (d, J=8.4 Hz, 1H), 7.00 (dd, J=9.5, 2.4 Hz, 1H);

HR-FABMS Calcd for C₁₃H₈FN₃O₃(M+H)⁺: 274.0622, Found: 274.0625.

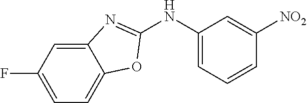 [SMILES: O=[N+]([O-])c1cccc(Nc2nc3cc(F)ccc3o2)c1]

The title compound was obtained in a similar manner to Example 1-1, except that 1-(5-fluoro-2-hydroxyphenyl)-3-(2-nitrophenyl)thiourea prepared according to Preparation Example 1-2 was used instead of 1-(5-fluoro-2-hydroxyphenyl)-3-(2-nitrophenyl)thiourea.
Yellow solid (77.6%);
m.p. 224˜226° C.;

¹H NMR (400 MHz, DMSO) δ 11.29 (s, 1H), 8.76 (t, J=2.2 Hz, 1H), 8.09 (dq, J=8.0, 1.1 Hz, 1H), 7.91 (dq, J=8.0, 1.1 Hz, 1H), 7.68 (t, J=8.0 Hz, 1H), 7.57 (dd, J=8.4, 4.4 Hz, 1H), 7.42 (dd, J=9.0, 2.6 Hz, 1H), 7.01 (ddd, J=9.8, 8.7, 2.7 Hz, 1H);

HR-FABMS Calcd for C₁₃H₈FN₃O₃(M+H)⁺: 274.0622, Found: 274.0624.

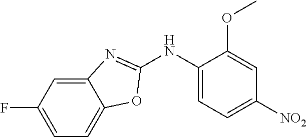 [SMILES: COc1cc([N+](=O)[O-])ccc1Nc1nc2cc(F)ccc2o1]

The title compound was obtained in a similar manner to Example 1-1, except that 1-(5-fluoro-2-hydroxyphenyl)-3-(2-methoxy-4-nitrophenyl)thiourea prepared according to Preparation Example 1-3 was used instead of 1-(5-fluoro-2-hydroxyphenyl)-3-(2-nitrophenyl)thiourea.
Yellow solid (78.6%);
m.p. 188˜190° C.;

¹H NMR (400 MHz, DMSO) δ 10.55 (s, 1H), 8.60 (d, J=9.2 Hz, 1H), 8.03 (dd, J=9.2, 2.8 Hz, 1H), 7.85 (d, J=2.4 Hz, 1H), 7.57 (dd, J=8.6, 4.6 Hz, 1H), 7.41 (dd, J=9.0, 2.6 Hz, 1H), 7.03 (ddd, J=10.2, 8.8, 2.6 Hz, 1H), 4.01 (s, 3H);

HR-FABMS Calcd for C₁₄H₁₀FN₃O₄(M+H)⁺: 304.0728, Found: 304.0731.

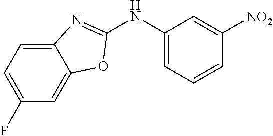 [SMILES: O=[N+]([O-])c1cccc(Nc2nc3ccc(F)cc3o2)c1]

The title compound was obtained in a similar manner to Example 1-1, except that 1-(4-fluoro-2-hydroxyphenyl)-3-(3-nitrophenyl)thiourea prepared according to Preparation Example 1-4 was used instead of 1-(5-fluoro-2-hydroxyphenyl)-3-(2-nitrophenyl)thiourea.
Yellow solid (75.0%);
m.p. 183˜184° C.;

¹H NMR (400 MHz, DMSO) δ 11.22 (s, 1H), 8.76 (t, J=2.2 Hz, 1H), 8.07 (dd, J=7.4, 1.0 Hz, 1H), 7.90 (dd, J=8.0, 2.0 Hz, 1H), 7.67 (t, J=8.2 Hz, 1H), 7.29 (dd, J=8.4, 2.4 Hz, 1H), 7.54 (dd, J=8.4, 5.2 Hz, 1H), 7.13 (ddd, J=10.2, 8.6, 2.4 Hz, 1H);

HR-FABMS Calcd for C₁₃H₈FN₃O₃(M+H)⁺: 274.0622, Found: 274.0624.

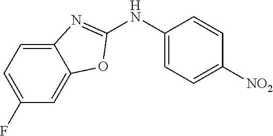 [SMILES: O=[N+]([O-])c1ccc(Nc2nc3ccc(F)cc3o2)cc1]

The title compound was obtained in a similar manner to Example 1-1, except that 1-(4-fluoro-2-hydroxyphenyl)-3-(4-nitrophenyl)thiourea prepared according to Preparation Example 1-5 was used instead of 1-(5-fluoro-2-hydroxyphenyl)-3-(2-nitrophenyl)thiourea.
Yellow solid (98.8%);
m.p. 255˜257° C.;

¹H NMR (400 MHz, DMSO) δ 11.47 (s, 1H), 8.31 (dt, J=9.2, 2.6 Hz, 2H), 7.96 (dt, J=9.2, 2.7 Hz, 2H), 7.61 (dd, J=8.8, 2.4 Hz, 1H), 7.55 (dd, J=8.8, 4.8 Hz, 1H), 7.15 (ddd, J=10.0, 8.8, 2.4 Hz, 1H);

HR-FABMS Calcd for C₁₃H₈FN₃O₃ (M+H)⁺: 274.0622, Found: 274.0625.

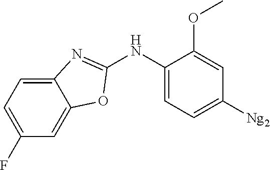

The title compound was obtained in a similar manner to Example 1-1, except that 1-(4-fluoro-2-hydroxyphenyl)-3-(2-methoxy-4-nitrophenyl)thiourea prepared according to Preparation Example 1-6 was used instead of 1-(5-fluoro-2-hydroxyphenyl)-3-(2-nitrophenyl)thiourea.
Yellow solid (76.5%);
m.p. 222˜225° C.;

¹H NMR (400 MHz, DMSO) δ 10.49 (s, 1H), 8.62 (d, J=8.8 Hz, 1H), 8.03 (dd, J=9.0, 2.6 Hz, 1H), 7.84 (d, J=2.8 Hz, 1H), 7.59 (dd, J=8.8, 2.4 Hz, 1H), 7.55 (dd, J=8.8, 4.8 Hz, 1H), 7.15 (ddd, J=10.2, 8.6, 2.4 Hz, 1H), 4.01 (s, 3H);

HR-FABMS Calcd for C₁₄H₁₀FN₃O₄(M+H)⁺: 304.0728, Found: 304.0732.

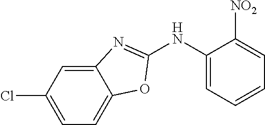 [SMILES: O=[N+]([O-])c1ccccc1Nc1nc2cc(Cl)ccc2o1]

The title compound was obtained in a similar manner to Example 1-1, except that 1-(5-chloro-2-hydroxyphenyl)-3-(2-nitrophenyl)thiourea prepared according to Preparation Example 1-7 was used instead of 1-(5-fluoro-2-hydroxyphenyl)-3-(2-nitrophenyl)thiourea.
Orange solid (80.9%);
m.p. 150˜152° C.;

¹H NMR (400 MHz, DMSO) δ 10.77 (s, 1H), 8.21 (s, 1H), 8.11 (d, J=8.4 Hz, 1H), 7.81 (t, J=7.4 Hz, 1H), 7.56 (d, J=8.8 Hz, 1H), 7.52 (s, 1H), 7.37 (d, J=7.6 Hz, 1H), 7.21 (dd, J=8.6, 2.2 Hz, 1H);

HR-FABMS Calcd for C₁₃H₈CIN₃O₃(M+H)⁺: 290.0327, Found: 290.0329.

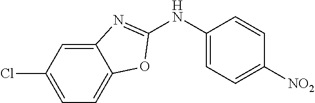 [SMILES: O=[N+]([O-])c1ccc(Nc2nc3cc(Cl)ccc3o2)cc1]

The title compound was obtained in a similar manner to Example 1-1, except that 1-(5-chloro-2-hydroxyphenyl)-3-(4-nitrophenyl)thiourea prepared according to Preparation Example 1-8 was used instead of 1-(5-fluoro-2-hydroxyphenyl)-3-(2-nitrophenyl)thiourea.
Yellow solid (84.0%);
m.p. 253˜255° C.;

¹H NMR (400 MHz, DMSO) δ 11.57 (s, 1H), 8.31 (dd, J=7.2, 2.0 Hz, 2H), 7.96 (dd, J=7.0, 2.2 Hz, 2H), 7.63 (d, J=2.0 Hz, 1H), 7.61 (d, J=8.8 Hz, 1H), 7.25 (dd, J=8.6, 2.2 Hz, 1H);

HR-FABMS Calcd for C₁₃H₈CIN₃O₃ (M+H)⁺: 290.0327, Found: 290.0329.

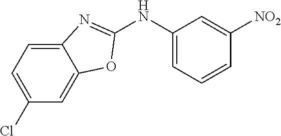 [SMILES: O=[N+]([O-])c1cccc(Nc2nc3ccc(Cl)cc3o2)c1]

The title compound was obtained in a similar manner to Example 1-1, except that 1-(4-chloro-2-hydroxyphenyl)-3-(3-nitrophenyl)thiourea prepared according to Preparation Example 1-9 was used instead of 1-(5-fluoro-2-hydroxyphenyl)-3-(2-nitrophenyl)thiourea.
Yellow solid (84.0%);
m.p. 235˜237° C.;

¹H NMR (400 MHz, DMSO) δ 11.33 (s, 1H), 8.74 (t, J=2.2 Hz, 1H), 8.08 (dq, J=8.4, 1.0 Hz, 1H), 7.91 (dq, J=8.0, 1.1 Hz, 1H), 7.76 (d, J=2.0 Hz, 1H), 7.61 (t, J=8.2 Hz, 1H), 7.54 (d, J=8.4 Hz, 1H), 7.31 (dd, J=8.0, 2.0 Hz, 1H);

HR-FABMS Calcd for C₁₃H₈CIN₃O₃ (M+H)⁺: 290.0327, Found: 290.0321.

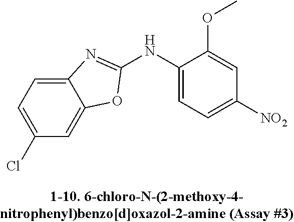 [SMILES: COc1cc([N+](=O)[O-])ccc1Nc1nc2ccc(Cl)cc2o1]

The title compound was obtained in a similar manner to Example 1-1, except that 1-(4-chloro-2-hydroxyphenyl)-3-(2-methoxy-4-nitrophenyl)thiourea prepared according to Preparation Example 1-10 was used instead of 1-(5-fluoro-2-hydroxyphenyl)-3-(2-nitrophenyl)thiourea.
Yellow solid (77.8%);
m.p. 209˜211° C.;

¹H NMR (400 MHz, DMSO) δ 10.57 (s, 1H), 8.61 (d, J=9.2 Hz, 1H), 8.02 (dd, J=9.2, 2.8 Hz, 1H), 7.85 (d, J=2.4 Hz, 1H), 7.76 (d, J=1.6 Hz, 1H), 7.55 (d, J=8.0 Hz, 1H), 7.33 (dd, J=8.6, 2.2 Hz, 1H), 4.01 (s, 3H);

HR-FABMS Calcd for C₁₄H₁₀CIN₃O₄ (M+H)⁺: 320.0433, Found: 320.0435.

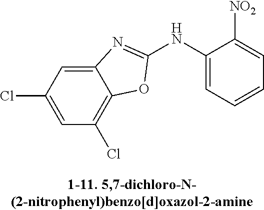 [SMILES: O=[N+]([O-])c1ccccc1Nc1nc2cc(Cl)cc(Cl)c2o1]

The title compound was obtained in a similar manner to Example 1-1, except that 1-(3,5-dichloro-2-hydroxyphenyl)-3-(2-nitrophenyl)thiourea prepared according to Preparation Example 1-11 was used instead of 1-(5-fluoro-2-hydroxyphenyl)-3-(2-nitrophenyl)thiourea.
Yellow solid (84.0%);
m.p. 199˜200° C.;

¹H NMR (400 MHz, DMSO) δ 11.06 (s, 1H), 8.11 (t, J=7.2 Hz, 1H), 8.10 (d, J=8.0 Hz, 1H), 7.82 (d, J=7.2 Hz, 1H), 7.52 (s, 1H), 7.43-7.39 (m, 2H);

HR-FABMS Calcd for C₁₃H₇Cl₂N₃O₃ (M+H)⁺: 323.9937, Found: 323.9937.

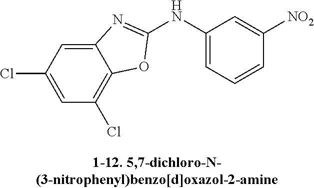 [SMILES: O=[N+]([O-])c1cccc(Nc2nc3cc(Cl)cc(Cl)c3o2)c1]

The title compound was obtained in a similar manner to Example 1-1, except that 1-(5-fluoro-2-hydroxyphenyl)-3-(3-nitrophenyl)thiourea prepared according to Preparation Example 1-12 was used instead of 1-(5-fluoro-2-hydroxyphenyl)-3-(2-nitrophenyl)thiourea.
Greenish brown solid (68.6%);
m.p. 208˜210° C.;

¹H NMR (400 MHz, DMSO) δ 11.66 (s, 1H), 8.73 (t, J=2.2 Hz, 1H), 8.04 (dd, J=7.8, 1.8 Hz, 1H), 7.94 (dq, J=8.2, 1.0 Hz, 1H), 7.70 (t, J=8.2 Hz, 1H), 7.64 (d, J=2.0 Hz, 1H), 7.43 (d, J=1.6 Hz, 1H);

HR-FABMS Calcd for C₁₃H₇Cl₂N₃O₃(M+H)⁺: 323.9937, Found: 323.9941.

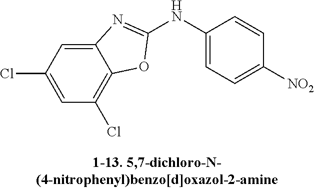 [SMILES: O=[N+]([O-])c1ccc(Nc2nc3cc(Cl)cc(Cl)c3o2)cc1]

The title compound was obtained in a similar manner to Example 1-1, except that 1-(3,5-dichloro-2-hydroxyphenyl)-3-(4-nitrophenyl)thiourea prepared according to Preparation Example 1-13 was used instead of 1-(5-fluoro-2-hydroxyphenyl)-3-(2-nitrophenyl)thiourea.
Greenish brown solid (49.2%);
m.p.>300° C.;

¹H NMR (400 MHz, DMSO) δ 11.87 (s, 1H), 8.32 (dt, J=9.2, 2.4 Hz, 2H), 7.94 (dd, J=7.2, 2.4 Hz, 2H), 7.64 (d, J=1.6 Hz, 1H), 7.45 (d, J=2.0 Hz, 1H);

HR-FABMS Calcd for C₁₃H₇Cl₂N₃O₃(M+H)⁺: 323.9937, Found: 323.9942.

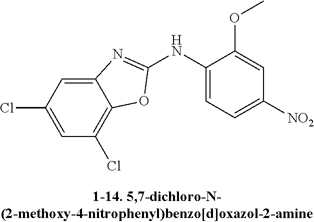 [SMILES: COc1cc([N+](=O)[O-])ccc1Nc1nc2cc(Cl)cc(Cl)c2o1]

The title compound was obtained in a similar manner to Example 1-1, except that 1-(3,5-dichloro-2-hydroxyphenyl)-3-(2-methoxy-4-nitrophenyl)thiourea prepared according to Preparation Example 1-14 was used instead of 1-(5-fluoro-2-hydroxyphenyl)-3-(2-nitrophenyl)thiourea.
Greenish brown solid (28.9%);
m.p. 232˜283° C.;

¹H NMR (400 MHz, CDCl₃) δ 8.61 (d, J=9.2 Hz, 1H), 8.06 (s, 1H), 8.05 (dd, J=9.2, 2.4 Hz, 1H), 7.82 (d, J=2.4 Hz, 1H), 7.45 (d, J=2.0 Hz, 1H), 7.21 (d, J=2.0 Hz, 1H), 4.08 (s, 3H);

HR-FABMS Calcd for C₁₄H₉Cl₂N₃O₄ (M+H)⁺: 354.0043, Found: 354.0044.

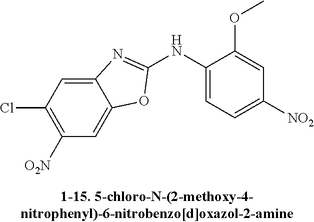 [SMILES: COc1cc([N+](=O)[O-])ccc1Nc1nc2cc(Cl)c([N+](=O)[O-])cc2o1]

The title compound was obtained in a similar manner to Example 1-1, except that 1-(5-chloro-2-hydroxy-4-nitrophenyl)-3-(2-methoxy-4-nitrophenyl)thiourea prepared according to Preparation Example 1-15 was used instead of 1-(5-fluoro-2-hydroxyphenyl)-3-(2-nitrophenyl)thiourea.
Yellow solid (37.0%);
m.p.>296° C.;

¹H-NMR (CDCl_{3,} 400 MHz) δ 8.63 (d, J=11.5 Hz, 1H), 8.14 (s, 1H), 8.06 (dd, J=11.5, 3 Hz, 1H), 8.04 (s, 1H), 7.85 (d, J=2.5 Hz, 1H), 7.67 (s, 1H), 4.09 (s, 3H);

HR-FABMS Calcd for C₁₄H₈CIN₄O₆ (M−H)⁻: 363.0138, Found: 363.0143.

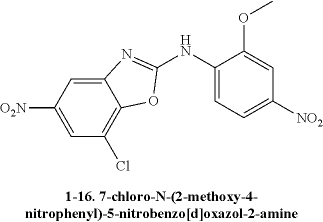 [SMILES: COc1cc([N+](=O)[O-])ccc1Nc1nc2cc([N+](=O)[O-])cc(Cl)c2o1]

The title compound was obtained in a similar manner to Example 1-1, except that 1-(3-chloro-2-hydroxy-5-nitrophenyl)-3-(2-methoxy-4-nitrophenyl)thiourea prepared according to Preparation Example 1-16 was used instead of 1-(5-fluoro-2-hydroxyphenyl)-3-(2-nitrophenyl)thiourea.
Yellow solid (78.6%);
m.p. 268˜271;

¹H NMR (400 MHz, CDCl₃) δ 8.64 (d, J=11.0 Hz, 1H), 8.34 (d, J=2.5 Hz, 1H), 8.20 (d, J=2.5 Hz, 1H), 8.16 (s, 1H), 8.07 (dd, J=11.0, 3.0 Hz, 1H), 7.85 (d, J=3.5 Hz, 1H), 4.10 (s, 3H);

HR-FABMS Calcd for C₁₄H₈CIN₄O₆ (M−H)⁻: 365.0114, Found: 365.0105.

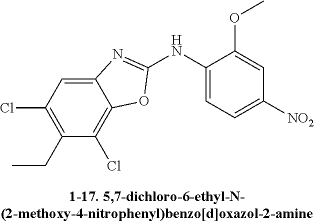 [SMILES: CCc1c(Cl)cc2nc(Nc3ccc([N+](=O)[O-])cc3OC)oc2c1Cl]

The title compound was obtained in a similar manner to Example 1-1, except that 1-(3,5-dichloro-4-ethyl-2-hydroxyphenyl)-3-(2-methoxy-4-nitrophenyl)thiourea prepared according to Preparation Example 1-17 was used instead of 1-(5-fluoro-2-hydroxyphenyl)-3-(2-nitrophenyl)thiourea.
Yellow solid (47.4%);
m.p. 226˜229° C.;

¹H NMR (400 MHz, CDCl₃) δ 8.61 (d, J=11.0 Hz, 1H), 8.05 (d, J=11.5, 3.0 Hz, 2H), 7.81 (d, J=3.0 Hz, 1H), 7.49 (s, 1H), 4.07 (s, 3H), 3.03 (d, J=18.5, 9.5 Hz, 2H), 1.25-1.19 (m, 3H);

HR-FABMS Calcd for C₁₆H₁₂C₁₂N₃O₄(M−H)⁻: 380.0210, Found: 380.0204.

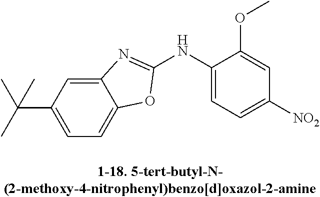 [SMILES: COc1cc([N+](=O)[O-])ccc1Nc1nc2cc(C(C)(C)C)ccc2o1]

The title compound was obtained in a similar manner to Example 1-1, except that 1-(5-tert-butyl-2-hydroxyphenyl)-3-(2-methoxy-4-nitrophenyl)thiourea prepared according to Preparation Example 1-18 was used instead of 1-(5-fluoro-2-hydroxyphenyl)-3-(2-nitrophenyl)thiourea.
Yellow solid (62.4%);
m.p. 150˜153° C.;

¹H NMR (400 MHz, DMSO) δ 10.35 (s, 1H), 8.64 (d, J=8.8 Hz, 1H), 8.00 (dd, J=9.2, 2.0 Hz, 1H), 7.80 (s, 1H), 7.53 (s, 1H), 7.41 (d, J=7.6 Hz, 1H), 7.22 (d, J=7.2 Hz, 1H), 3.99 (s, 3H), 1.33 (s, 9H);

HR-FABMS Calcd for C₁₈H₂₀N₃O₄(M+H)⁺: 342.1448, Found: 342.1451.

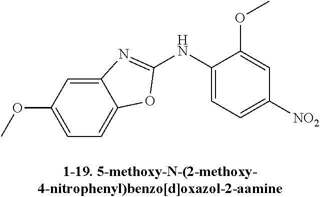 [SMILES: COc1ccc2oc(Nc3ccc([N+](=O)[O-])cc3OC)nc2c1]

The title compound was obtained in a similar manner to Example 1-1, except that 1-(2-hydroxy-5-methoxyphenyl)-3-(2-methoxy-4-nitrophenyl)thiourea prepared according to Preparation Example 1-19 was used instead of 1-(5-fluoro-2-hydroxyphenyl)-3-(2-nitrophenyl)thiourea.
Yellow solid (55.0%);
m.p. 148˜152° C.;

¹H NMR (400 MHz, CDCl₃) δ 8.65 (d, J=9.2 Hz, 1H), 8.04 (dd, J=8.8, 2.4 Hz, 1H), 7.90 (s, 1H), 7.78 (d, J=2.4 Hz, 1H), 7.12 (d, J=2.8 Hz, 1H), 6.78 (dd, J=8.8, 2.8 Hz, 1H), 4.06 (s, 3H), 3.99 (s, 1H), 3.86 (s, 3H);

HR-FABMS Calcd for C₁₅H₁₄N₃O₅(M+H)⁺: 316.0928, Found: 316.0928.

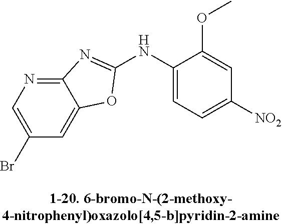 [SMILES: COc1cc([N+](=O)[O-])ccc1Nc1nc2ncc(Br)cc2o1]

The title compound was obtained in a similar manner to Example 1-1, except that 1-(5-bromo-3-hydroxypyrdin-2-yl)-3-(2-methoxy-4-nitrophenyl)thiourea prepared according to Preparation Example 1-20 was used instead of 1-(5-fluoro-2-hydroxyphenyl)-3-(2-nitrophenyl)thiourea.
Yellow solid (32.1%);
m.p. 216˜219° C.;

¹H NMR (400 MHz, CDCl₃) δ 8.77 (d, J=8.8 Hz, 1H), 8.49 (d, J=2.0 Hz, 1H), 8.07 (s, 1H), 8.04 (dd, J=9.0 Hz, 1H), 7.83 (d, J=2.4 Hz, 1H), 7.80 (d, J=2.0 Hz, 1H), 4.09 (s, 3H);

HR-FABMS Calcd for C₁₃H₁₀BrN₄O₄(M+H)⁺: 364.9880, Found: 364.9885

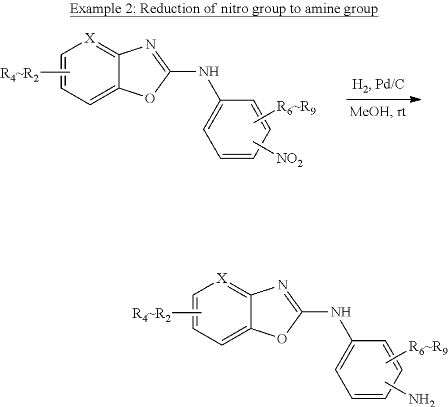

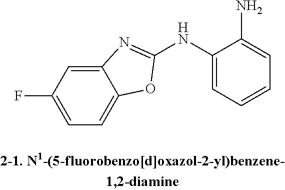 [SMILES: Nc1ccccc1Nc1nc2cc(F)ccc2o1]

1 equivalent of 5-fluoro-N-(2-nitrophenyl)benzo[d]oxazol-2-amine prepared according to Example 1-1 was dissolved in methanol, and then an appropriate amount of Pd/C was added to prepare a mixture. The mixture was simultaneously stirred and reacted at room temperature for 15 to 30 minutes under hydrogen substitution, and then filtered to remove Pd/C. A solvent was removed by evaporation under reduced pressure, and thus the title compound was obtained.
Pale brown solid (55.9%);
m.p. 163˜165° C.;

¹H NMR (400 MHz, Acetone-d₆) δ 8.50 (s, 1H), 7.57 (dd, J=8.0, 1.6 Hz, 1H), 7.32 (dd, J=8.8, 4.4 Hz, 1H), 7.05 (dd, J=9.0, 2.6 Hz, 1H), 7.00 (td, J=7.7, 1.4, 1H), 6.88 (dd, J=8.0, 1.2 Hz, 1H), 6.81 (ddd, J=10.0, 8.5, 2.7 Hz, 1H), 6.71 (td, J=7.7, 1.2 Hz, 1H), 4.75 (s, 2H);

HR-FABMS Calcd for C₁₃H₁₀FN₃O (M+H)⁺: 244.0881, Found: 244.0882.

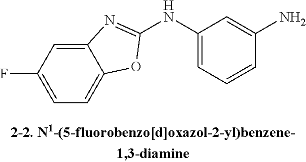 [SMILES: Nc1cccc(Nc2nc3cc(F)ccc3o2)c1]

The title compound was obtained in a similar manner to Example 2-1, except that 5-fluoro-N-(3-nitrophenyl)benzo[d]oxazol-2-amine prepared according to Example 1-2 was used instead of 5-fluoro-N-(2-nitrophenyl)benzo[d]oxazol-2-amine.
Pale brown solid (41.0%);
m.p. 151˜152° C.;

¹H NMR (400 MHz, CDCl₃) δ 7.23 (dd, J=8.8, 4.4 Hz, 1H), 7.19-7.13 (m, 4H), 6.85-6.79 (m, 2H), 6.45 (dq, J=8.0, 0.9 Hz, 1H), 3.81 (s, 2H);

HR-FABMS Calcd for C₁₃H₁₀FN₃O (M+H)⁺: 244.0881, Found: 244.0881.

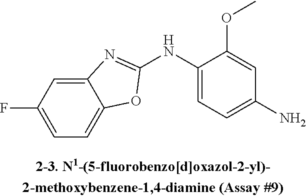 [SMILES: COc1cc(N)ccc1Nc1nc2cc(F)ccc2o1]

The title compound was obtained in a similar manner to Example 2-1, except that 5-fluoro-N-(2-methoxy-4-nitrophenyl)benzo[d]oxazol-2-amine prepared according to Example 1-3 was used instead of 5-fluoro-N-(2-nitrophenyl)benzo[d]oxazol-2-amine.
Dark brown solid (61.7%);
m.p. 112˜114° C.;

¹H NMR (400 MHz, Acetone-d₆) δ 8.28 (s, 1H), 7.84 (dd, J=8.6, 1.8 Hz, 1H), 7.29 (dd, J=8.8, 4.4 Hz, 1H), 7.07 (dd, J=9.0, 2.6 Hz, 1H), 6.80 (ddd, J=10.0, 8.6, 2.6 Hz, 1H), 6.45 (d, J=2.4 Hz, 1H), 6.33 (dd, J=8.8, 2.4 Hz, 1H), 4.58 (s, 2H), 3.81 (s, 3H);

HR-FABMS Calcd for C₁₄H₁₂FN₃O₂ (M+H)⁺: 274.0986, Found: 274.0985.

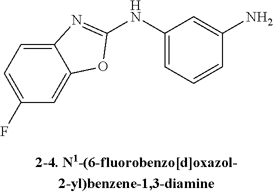 [SMILES: Nc1cccc(Nc2nc3ccc(F)cc3o2)c1]

The title compound was obtained in a similar manner to Example 2-1, except that 6-fluoro-N-(3-nitrophenyl)benzo[d]oxazol-2-amine prepared according to Example 1-4 was used instead of 5-fluoro-N-(2-nitrophenyl)benzo[d]oxazol-2-amine.
Pale brown solid (68.0%);
m.p. 176˜177° C.;

¹H NMR (400 MHz, Acetone-d₆) δ 9.24 (s, 1H), 7.37 (dd, J=8.8, 4.8 Hz, 1H), 7.25 (d, J=2.4, 1H), 7.23-7.22 (m, 1H), 7.06-6.98 (m, 3H), 6.39 (dt, J=7.6, 1.9 Hz, 1H), 4.70 (s, 2H);

HR-FABMS Calcd for C₁₃H₁₀FN₃O (M+H)⁺: 244.0881, Found: 244.0883.

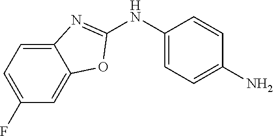 [SMILES: Nc1ccc(Nc2nc3ccc(F)cc3o2)cc1]

The title compound was obtained in a similar manner to Example 2-1, except that 6-fluoro-N-(4-nitrophenyl)benzo[d]oxazol-2-amine prepared according to Example 1-5 was used instead of 5-fluoro-N-(2-nitrophenyl)benzo[d]oxazol-2-amine.
Gray solid (50.2%);
m.p. 164˜166° C.;

¹H NMR (400 MHz, Acetone-d₆) δ 9.04 (s, 1H), 7.50 (dt, J=8.8, 2.6 Hz, 2H), 7.31 (dd, J=8.6, 5.0 Hz, 1H), 7.20 (dd, J=8.4, 2.4 Hz, 1H), 6.97 (ddd, J=10.0, 8.6, 2.6 Hz, 1H), 6.70 (dt, J=8.8, 2.7 Hz, 2H), 4.47 (s, 2H);

HR-FABMS Calcd for C₁₃H₁₀FN₃O (M+H)⁺: 244.0881, Found: 244.0882.

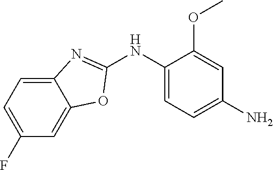 [SMILES: COc1cc(N)ccc1Nc1nc2ccc(F)cc2o1]

The title compound was obtained in a similar manner to Example 2-1, except that 6-fluoro-N-(2-methoxy-4-nitrophenyl)benzo[d]oxazol-2-amine prepared according to Example 1-6 was used instead of 5-fluoro-N-(2-nitrophenyl)benzo[d]oxazol-2-amine.
Gray solid (57.2%);
m.p. 168˜170° C.;

¹H NMR (400 MHz, Acetone-d₆) δ 8.19 (s, 1H), 7.89 (d, J=8.8 Hz, 1H), 7.29 (dd, J=8.4, 5.2 Hz, 1H), 7.19 (dd, J=8.4, 2.4 Hz, 1H), 6.97 (ddd, J=9.9, 8.7, 2.5 Hz, 1H), 6.45 (d, J=2.4 Hz, 1H), 6.33 (dd, J=8.2, 2.2 Hz, 1H), 4.55 (s, 2H), 3.81 (s, 3H);

HR-FABMS Calcd for C₁₄H₁₂FN₃O₂ (M+H)⁺: 274.0986, Found: 274.0982.

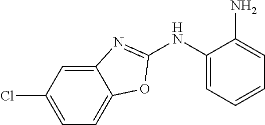 [SMILES: Nc1ccccc1Nc1nc2cc(Cl)ccc2o1]

The title compound was obtained in a similar manner to Example 2-1, except that 5-chloro-N-(2-nitrophenyl)benzo[d]oxazol-2-amine prepared according to Example 1-7 was used instead of 5-fluoro-N-(2-nitrophenyl)benzo[d]oxazol-2-amine.
Brown solid (60.5%);
m.p. 216-217° C.;

¹H NMR (400 MHz, DMSO-d₆) δ12.56 (s, 2H), 10.56 (s, 1H), 7.43 (d, J=6.4 Hz, 1H), 7.40-7.36 (m, 2H), 7.24-7.21 (m, 2H), 7.07 (d, J=7.2 Hz, 1H), 6.93 (td, J=7.6, 1.2 Hz, 1H);

HR-FABMS Calcd for C₁₃H₁₀CIN₃O (M+H)⁺: 260.0585, Found: 260.0580.

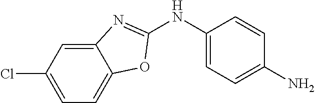 [SMILES: Nc1ccc(Nc2nc3cc(Cl)ccc3o2)cc1]

The title compound was obtained in a similar manner to Example 2-1, except that 5-chloro-N-(4-nitrophenyl)benzo[d]oxazol-2-amine prepared according to Example 1-8 was used instead of 5-fluoro-N-(2-nitrophenyl)benzo[d]oxazol-2-amine.
Grey solid (62.6%);
m.p. 211˜213° C.;

¹H NMR (400 MHz, CDCl₃) δ 7.40 (d, J=2.0 Hz 1H), 7.33 (d, J=8.4 Hz, 2H), 7.19 (d, J=8.4 Hz, 1H), 7.05 (dd, J=8.6, 2.2 Hz, 1H), 6.87 (s, 1H), 6.73 (d, J=8.4 Hz, 2H), 3.65 (s, 2H);

HR-FABMS Calcd for C₁₃H₁₀CIN₃O (M+H)⁺: 260.0585, Found: 260.0587.

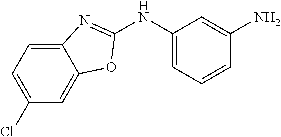 [SMILES: Nc1cccc(Nc2nc3ccc(Cl)cc3o2)c1]

The title compound was obtained in a similar manner to Example 2-1, except that 6-chloro-N-(3-nitrophenyl)benzo[d]oxazol-2-amine prepared according to Example 1-9 was used instead of 5-fluoro-N-(2-nitrophenyl)benzo[d]oxazol-2-amine.
Pale brown solid (56.2%);
m.p. 181˜183° C.;

¹H NMR (400 MHz, CDCl₃) δ 7.38 (d, J=8.4 Hz, 1H), 7.34 (d, J=2.0, 1H), 7.21 (dd, J=8.4, 2.0 Hz, 1H), 7.16-7.12 (m, 2H), 7.00 (s, 1H), 6.80 (dt, J=8.0, 1.0 Hz, 1H), 6.44 (dt, J=8.0, 1.2 Hz, 1H), 3.82 (s, 2H);

HR-FABMS Calcd for C₁₃H₁₀CIN₃O (M+H)⁺: 260.0585, Found: 260.0583.

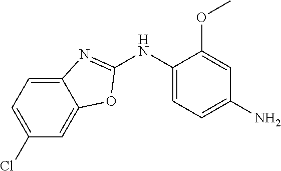 [SMILES: COc1cc(N)ccc1Nc1nc2ccc(Cl)cc2o1]

The title compound was obtained in a similar manner to Example 2-1, except that 6-chloro-N-(2-methoxy-4-nitrophenyl)benzo[d]oxazol-2-amine prepared according to Example 1-10 was used instead of 5-fluoro-N-(2-nitrophenyl)benzo[d]oxazol-2-amine.
Dark gray solid (33.0%);
m.p. 188˜189° C.;

¹H NMR (400 MHz, Acetone-d₆) δ 8.33 (d, J=8.4 Hz, 1H), 7.80 (s, 1H), 7.46 (d, J=1.6 Hz, 1H), 7.39 (d, J=8.4 Hz, 1H), 7.24 (dd, J=8.6, 2.2 Hz, 1H), 6.52 (d, J=2.0 Hz, 1H), 6.42 (dd, J=8.4, 2.0 Hz, 1H), 3.91 (s, 3H), 3.79 (s, 2H);

HR-FABMS Calcd for C₁₄H₁₂CIN₃O₂ (M+H)⁺: 290.0691, Found: 290.0687.

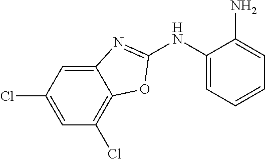 [SMILES: Nc1ccccc1Nc1nc2cc(Cl)cc(Cl)c2o1]

The title compound was obtained in a similar manner to Example 2-1, except that 5,7-dichloro-N-(2-nitrophenyl)benzo[d]oxazol-2-amine prepared according to Example 1-11 was used instead of 5-fluoro-N-(2-nitrophenyl)benzo[d]oxazol-2-amine.
Pale brown solid (46.0%);
m.p. 220˜222° C.;

¹H NMR (400 MHz, Acetone-d₆) δ 7.81 (dd, J=7.8, 1.0 Hz, 1H), 7.43 (d, J=2.0 Hz, 1H), 7.23 (d, J=1.6 Hz, 1H), 6.89 (td, J=7.6, 1.2 Hz, 1H), 6.77 (t, J=7.2 Hz, 1H), 6.64 (d, J=8.0 Hz, 1H), 5.76 (s, 2H);

HR-FABMS Calcd for C₁₃H₉Cl₂N₃O (M+H)⁺: 294.0195, Found: 294.0196.

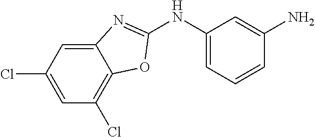 [SMILES: Nc1cccc(Nc2nc3cc(Cl)cc(Cl)c3o2)c1]

The title compound was obtained in a similar manner to Example 2-1, except that 5,7-dichloro-N-(3-nitrophenyl)benzo[d]oxazol-2-amine prepared according to Example 1-12 was used instead of 5-fluoro-N-(2-nitrophenyl)benzo[d]oxazol-2-amine.
Brown solid (58.7%);
m.p. 199˜200° C.;

¹H NMR (400 MHz, CDCl₃) δ 7.35 (d, J=2.0 Hz, 1H), 7.17-7.12 (m, 3H), 7.08 (s, 1H), 6.80 (dd, J=8.0, 1.2 Hz, 1H), 6.46 (dt, J=8.4, 1.0 Hz, 1H), 3.81 (s, 2H);

HR-FABMS Calcd for C₁₃H₉Cl₂N₃O (M+H)⁺: 294.0195, Found: 294.0195.

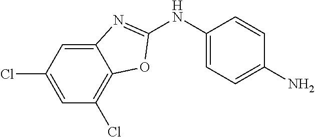 [SMILES: Nc1ccc(Nc2nc3cc(Cl)cc(Cl)c3o2)cc1]

The title compound was obtained in a similar manner to Example 2-1, except that 5,7-dichloro-N-(4-nitrophenyl)benzo[d]oxazol-2-amine prepared according to Example 1-13 was used instead of 5-fluoro-N-(2-nitrophenyl)benzo[d]oxazol-2-amine.
Gray solid (38.4%);
m.p. 204˜206° C.;

¹H NMR (400 MHz, DMSO-d₆) δ 10.58 (s, 1H), 7.40 (d, J=2.0 Hz, 1H), 7.34 (dd, J=8.8, 2.2, 2H), 7.26 (d, J=2.0 Hz, 1H), 6.60 (dt, J=8.8, 2.6 Hz, 2H), 5.12 (s, 2H);

HR-FABMS Calcd for C₁₃H₉Cl₂N₃O (M+H)⁺: 294.0195, Found: 294.0199.

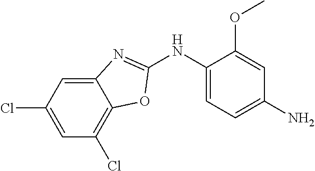 [SMILES: COc1cc(N)ccc1Nc1nc2cc(Cl)cc(Cl)c2o1]

The title compound was obtained in a similar manner to Example 2-1, except that 5,7-dichloro-N-(2-methoxy-4-nitrophenyl)benzo[d]oxazol-2-amine prepared according to Example 1-14 was used instead of 5-fluoro-N-(2-nitrophenyl)benzo[d]oxazol-2-amine.
Gray solid (45.2%);
m.p. 150˜152° C.;

¹H NMR (400 MHz, Acetone-d₆) δ 8.66 (s, 1H), 7.79 (d, J=8.8 Hz, 1H), 7.28 (d, J=2.0 Hz, 1H), 7.12 (d, J=1.6 Hz, 1H), 6.46 (d, J=2.4 Hz, 1H), 6.33 (dd, J=8.6, 2.2 Hz, 1H), 4.64 (s, 2H), 3.82 (s, 3H);

HR-FABMS Calcd for C₁₄H₁₁Cl₂N₃O₂ (M+H)⁺: 324.0301, Found: 324.0296.

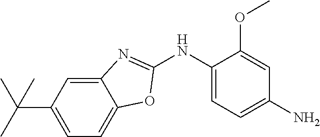 [SMILES: COc1cc(N)ccc1Nc1nc2cc(C(C)(C)C)ccc2o1]

The title compound was obtained in a similar manner to Example 2-1, except that 5-tert-butyl-N-(2-methoxy-4-nitrophenyl)benzo[d]oxazol-2-amine prepared according to Example 1-18 was used instead of 5-fluoro-N-(2-nitrophenyl)benzo[d]oxazol-2-amine.
Brown solid (68.8%);
m.p. 185˜188° C.;

¹H NMR (400 MHz, DMSO-d₆) δ 8.98 (s, 1H), 7.35 (d, J=8.0 Hz, 1H), 7.28 (d, J=1.6 Hz, 1H), 7.23 (d, J=8.4 Hz, 1H), 7.03 (dd, J=8.8, 1.6 Hz, 1H), 6.31 (d, J=2.4 Hz, 1H), 6.17 (dd, J=8.4, 2.4 Hz, 1H), 5.04 (s, 2H), 3.70 (s, 3H), 1.29 (s, 9H);

HR-FABMS Calcd for C₁₈H₂₂N₃O₂ (M+H)⁺: 312.1707, Found: 312.1707.

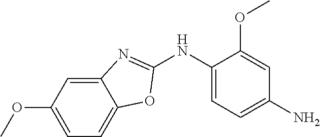 [SMILES: COc1ccc2oc(Nc3ccc(N)cc3OC)nc2c1]

The title compound was obtained in a similar manner to Example 2-1, except that 5-methoxy-N-(2-methoxy-4-nitrophenyl)benzo[d]oxazol-2-amine prepared according to Example 1-19 was used instead of 5-fluoro-N-(2-nitrophenyl)benzo[d]oxazol-2-amine.
Black oil (51.9%);

¹H NMR (400 MHz, DMSO-d₆) 9.02 (s, 1H), 7.30 (d, J=8.0 Hz, 1H), 7.22 (d, J=8.4 Hz, 1H), 6.84 (d, J=2.8 Hz, 1H), 6.55 (dd, J=8.8, 2.4 Hz, 1H), 6.31 (d, J=2.4 Hz, 1H), 6.61 (dd, J=8.0, 2.4 Hz, 1H), 5.05 (s, 2H), 3.73 (s, 3H), 3.70 (s, 3H);

HR-FABMS Calcd for C₁₅H₁₆N₃O₃(M+H)⁺: 286.1186, Found: 286.1189.

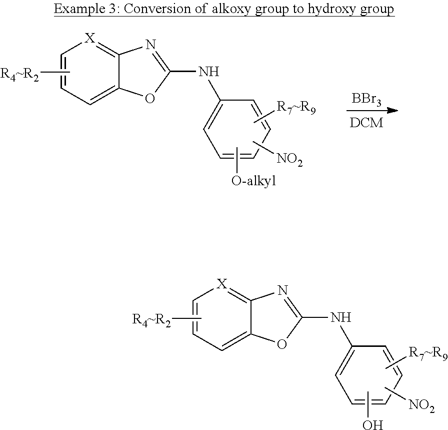

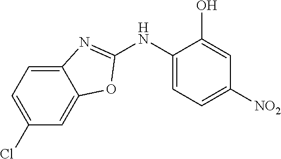 [SMILES: O=[N+]([O-])c1ccc(Nc2nc3ccc(Cl)cc3o2)c(O)c1]

1 equivalent of 6-chloro-N-(2-methoxy-4-nitrophenyl)benzo[d]oxazol-2-amine prepared according to Example 1-10 was mixed with 10 mL of dichloromethane, 3 equivalents of boron trifluoride (BBr₃) was added thereto under nitrogen gas substitution to prepare a mixture, and then the mixture was stirred at 0° C. for 24 hours. Then, the stirred mixture was diluted with cold water, extracted with ethyl acetate, and dried with magnesium sulfate. The product was evaporated under reduced pressure to remove a solvent, and then purified by column chromatography to obtain the title compound.
Yellow solid (33.5%);
m.p. 218˜219° C.;

¹H NMR (CDCl_{3,} 400 MHz) δ 8.13 (d, J=9.2 Hz, 1H), 7.95 (dd, J=8.8, 2.4 Hz, 1H), 7.84 (d, J=2.4 Hz, 1H), 7.45 (d, J=8.0 Hz, 1H) 7.42 (d, J=1.6 Hz, 1H), 7.29 (dd, J=8.8, 2.0 Hz, 1H);

HR-FABMS Calcd for C₁₃H₈CIN₃O₄ (M+H)⁺: 306.6813.

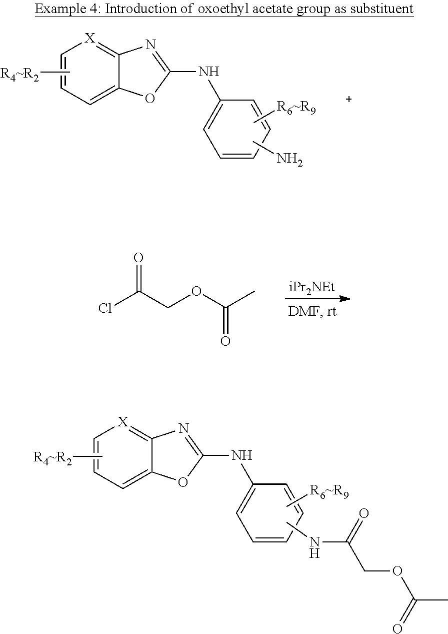

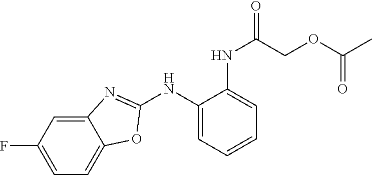 [SMILES: CC(=O)OCC(=O)Nc1ccccc1Nc1nc2cc(F)ccc2o1]

1 equivalent of N¹-(5-fluorobenzo[d]oxazol-2-yl)benzene-1,4-diamine prepared according to Example 2-1 was mixed with dimethylformamide, 2-chloro-2-oxoethyl acetate and diisopropylethylamine were added thereto at each 1 equivalent to prepare a mixture, and the mixture was stirred at room temperature for 16 hours to react. After terminating the reaction by adding 10% HCl aqueous solution, the reaction product was extracted with ethyl acetate, and an organic layer is washed with saturated aqueous sodium bicarbonate solution and saturated aqueous sodium chloride solution. This reaction product was dried with anhydrous magnesium sulfate and filtered, and the solvent is concentrated under reduced pressure to obtain the title compound.
Pale brown solid (34.2%);
m.p. 149˜150° C.;

¹H NMR (400 MHz, Acetone-d₆) δ 9.30 (s, 1H), 9.01 (s, 1H), 8.05 (d, J=8.4 Hz, 1H), 7.59 (dt, J=8.4, 2.1 Hz, 1H), 7.39 (dd, J=8.8, 4.4 Hz, 1H), 7.32 (td, J=7.8, 1.6 Hz, 1H), 7.21 (td, J=7.7, 1.4 Hz, 1H), 7.15 (dd, J=9.0, 2.6 Hz, 1H), 6.89 (ddd, J=9.8, 8.7, 2.7 Hz, 1H), 4.73 (s, 2H), 2.05 (s, 3H);

HR-FABMS Calcd for C₁₇H₁₄FN₃O₄(M+H)⁺: 344.1046, Found: 344.1042.

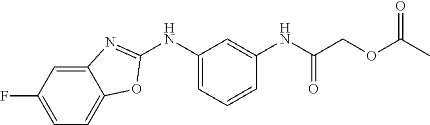 [SMILES: CC(=O)OCC(=O)Nc1cccc(Nc2nc3cc(F)ccc3o2)c1]

The title compound was obtained in a similar manner to Example 4-1, except that N¹-(5-fluorobenzo[d]oxazol-2-yl)benzene-1,3-diamine prepared according to Example 2-2 was used instead of N¹-(5-fluorobenzo[d]oxazol-2-yl)benzene-1,4-diamine.
Pale brown solid (81.8%);
m.p. 187˜189° C.;

¹H NMR (400 MHz, Acetone-d₆) δ 9.62 (s, 1H), 9.29 (s, 1H), 8.09 (t, J=1.6 Hz, 1H), 7.65 (dq, J=7.8, 1.1 Hz, 1H), 7.40-7.31 (m, 3H), 7.17 (dd, J=9.0, 2.4 Hz, 1H), 6.89 (ddd, J=9.6, 8.7, 2.5 Hz, 1H), 4.70 (s, 2H), 2.14 (s, 3H);

HR-FABMS Calcd for C₁₇H₁₄FN₃O₄(M+H)⁺: 344.1046, Found: 344.1044.

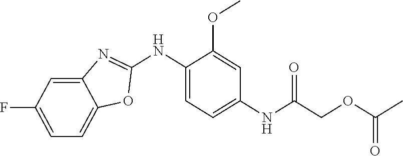 [SMILES: COc1cc(NC(=O)COC(C)=O)ccc1Nc1nc2cc(F)ccc2o1]

The title compound was obtained in a similar manner to Example 4-1, except that N¹-(5-fluorobenzo[d]oxazol-2-yl)-2-methoxybenzene-1,4-diamine prepared according to Example 2-3 was used instead of N¹-(5-fluorobenzo[d]oxazol-2-yl)benzene-1,4-diamine.
Brown solid (57.3%);
m.p. 145˜147° C.;

¹H NMR (400 MHz, Acetone-d₆) δ 9.22 (s, 1H), 8.71 (s, 1H), 8.33 (d, J=8.8 Hz, 1H), 7.65 (d, J=2.4 Hz, 1H), 7.37 (dd, J=8.4, 4.4 Hz, 1H), 7.18-7.14 (m, 2H), 6.88 (ddd, J=9.8, 8.7, 2.7 Hz, 1H), 4.68 (s, 2H), 3.91 (s, 3H), 2.13 (s, 3H);

HR-FABMS Calcd for C₁₈H₁₆FN₃O₄ (M+H)⁺: 374.1147, Found: 374.1154.

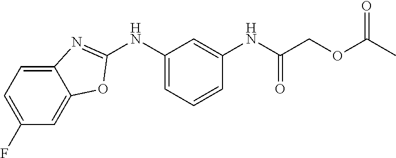 [SMILES: CC(=O)OCC(=O)Nc1cccc(Nc2nc3ccc(F)cc3o2)c1]

The title compound was obtained in a similar manner to Example 4-1, except that N¹-(6-fluorobenzo[d]oxazol-2-yl)benzene-1,3-diamine prepared according to Example 2-4 was used instead of N¹-(5-fluorobenzo[d]oxazol-2-yl)benzene-1,4-diamine.
Pale brown solid (71.8%);
m.p. 201˜203° C.;

¹H NMR (400 MHz, Acetone-d₆) δ 9.56 (s, 1H), 9.29 (s, 1H), 8.09 (t, J=2.2 Hz, 1H), 7.65 (ddd, J=7.6, 2.2, 1.4 Hz, 1H), 7.41 (dd, J=8.8, 4.8 Hz, 1H), 7.36 (dt, J=8.8, 1.6 Hz, 1H), 7.32 (t, J=8.0 Hz, 1H), 7.28 (dd, J=8.2, 2.6 Hz, 1H), 7.03 (ddd, J=10.0, 8.7, 2.6 Hz, 1H), 4.69 (s, 2H), 2.13 (s, 3H);

HR-FABMS Calcd for C₁₇H₁₄FN₃O₄(M+H)⁺: 344.1046, Found: 344.1044.

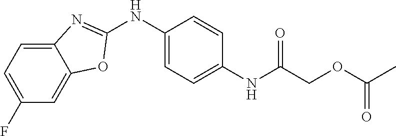 [SMILES: CC(=O)OCC(=O)Nc1ccc(Nc2nc3ccc(F)cc3o2)cc1]

The title compound was obtained in a similar manner to Example 4-1, except that N¹-(6-fluorobenzo[d]oxazol-2-yl)benzene-1,4-diamine prepared according to Example 2-5 was used instead of N¹-(5-fluorobenzo[d]oxazol-2-yl)benzene-1,4-diamine.
Pale brown solid (76.6%);
m.p. 215˜216° C.;

¹H NMR (400 MHz, Acetone-d₆) δ 9.50 (s, 1H), 9.18 (s, 1H), 7.79 (dt, J=9.2, 2.5 Hz, 2H), 7.66 (dt, J=8.8, 2.4 Hz, 2H), 7.40 (dd, J=8.6, 5.0 Hz, 1H), 7.26 (dd, J=8.4, 2.4 Hz, 1H), 7.02 (ddd, J=10.0, 8.6, 2.6 Hz, 1H), 4.67 (s, 2H), 2.13 (s, 3H);

HR-FABMS Calcd for C₁₇H₁₄FN₃O₄ (M+H)⁺: 344.1046, Found: 344.1049.

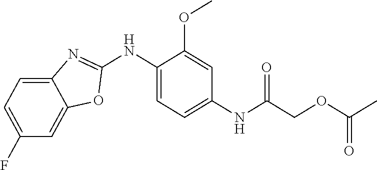 [SMILES: COc1cc(NC(=O)COC(C)=O)ccc1Nc1nc2ccc(F)cc2o1]

The title compound was obtained in a similar manner to Example 4-1, except that N¹-(6-fluorobenzo[d]oxazol-2-yl)-2-methoxybenzene-1,4-diamine prepared according to Example 2-6 was used instead of N¹-(5-fluorobenzo[d]oxazol-2-yl)benzene-1,4-diamine.
White solid (78.8%);
m.p. 181˜182° C.;

¹H NMR (400 MHz, Acetone-d₆) δ 9.21 (s, 1H), 8.65 (s, 1H), 8.35 (d, J=8.8 Hz, 1H), 7.64 (d, J=2.0 Hz, 1H), 7.39 (dd, J=8.4, 4.8 Hz, 1H), 7.26 (dd, J=8.2, 2.6 Hz, 1H), 7.16 (dd, J=8.6, 2.2 Hz, 1H), 7.02 (ddd, J=10.0, 8.6, 2.6 Hz, 1H), 4.68 (s, 2H), 3.91 (s, 3H), 2.13 (s, 3H);

HR-FABMS Calcd for C₁₈H₁₆FN₃O₅ (M+H)⁺: 374.1147, Found: 374.1149.

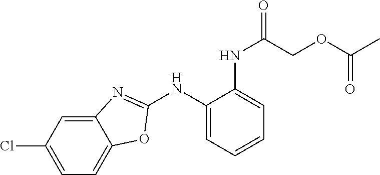 [SMILES: CC(=O)OCC(=O)Nc1ccccc1Nc1nc2cc(Cl)ccc2o1]

The title compound was obtained in a similar manner to Example 4-1, except that N¹-(5-chlorobenzo[d]oxazol-2-yl)benzene-1,2-diamine prepared according to Example 2-7 was used instead of N¹-(5-fluorobenzo[d]oxazol-2-yl)benzene-1,4-diamine.
Pale brown solid (38.0%);
m.p. 95˜97° C.;

¹H NMR (400 MHz, Acetone-d₆) δ 9.30 (s, 1H), 9.05 (s, 1H), 8.05 (d, J=8.8 Hz, 1H), 7.62 (dt, J=7.6, 2.8 Hz, 1H), 7.41 (d, J=8.4 Hz, 2H), 7.33 (d, J=0.4 Hz, 1H), 7.22 (td, J=7.7, 1.4 Hz, 1H), 7.14 (dd, J=8.4, 2.4 Hz, 1H), 4.73 (s, 2H), 2.05 (s, 3H);

HR-FABMS Calcd for C₁₇H₁₄CIN₃O₄(M+H)⁺: 360.0751, Found: 360.0756.

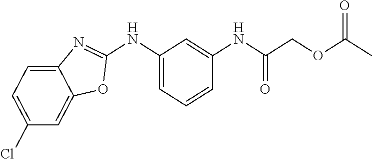 [SMILES: CC(=O)OCC(=O)Nc1cccc(Nc2nc3ccc(Cl)cc3o2)c1]

The title compound was obtained in a similar manner to Example 4-1, except that N¹-(6-chlorobenzo[d]oxazol-2-yl)benzene-1,3-diamine prepared according to Example 2-9 was used instead of N¹-(5-fluorobenzo[d]oxazol-2-yl)benzene-1,4-diamine.
Pale brown solid (82.5%);
m.p. 211-212° C.;

¹H NMR (400 MHz, Acetone-d₆) δ 9.63 (s, 1H), 9.30 (s, 1H), 8.09 (t, J=1.8 Hz, 1H), 7.65 (dq, J=8.0, 1.2 Hz, 1H), 7.49 (d, J=2.4 Hz, 1H), 7.41 (d, J=8.0 Hz, 1H), 7.37 (dq, J=7.6, 1.4 Hz, 1H), 7.33 (t, J=8.0 Hz, 1H), 7.26 (dd, J=8.4, 2.0 Hz, 1H), 4.70, (s, 2H) 2.13 (s, 3H);

HR-FABMS Calcd for C₁₇H₁₄CIN₃O₄ (M+H)⁺: 360.0751, Found: 360.0747.

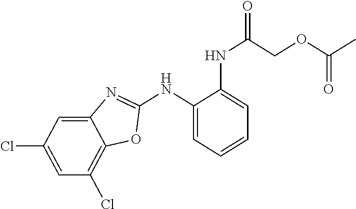 [SMILES: CC(=O)OCC(=O)Nc1ccccc1Nc1nc2cc(Cl)cc(Cl)c2o1]

The title compound was obtained in a similar manner to Example 4-1, except that N¹-(5,7-dichlorobenzo[d]oxazol-2-yl)benzene-1,2-diamine prepared according to Example 2-11 was used instead of N¹-(5-fluorobenzo[d]oxazol-2-yl)benzene-1,4-diamine.
Pale brown solid (85.3%);
m.p. 98˜100° C.;

¹H NMR (400 MHz, Acetone-d₆) δ 9.28 (s, 2H), 8.06 (d, J=8.0 Hz, 1H), 7.59 (dd, J=8.0, 1.6 Hz, 1H), 7.37 (d, J=2.0 Hz, 1H), 7.34 (td, J=7.8, 1.2 Hz, 1H), 7.24 (td, J=7.8, 1.4 Hz, 1H), 7.22 (d, J=1.6 Hz, 1H), 4.73 (s, 2H), 2.07 (s, 3H);

HR-FABMS Calcd for C₁₇H₁₃Cl₂N₃O₄ (M+H)⁺: 394.0361, Found: 394.0360.

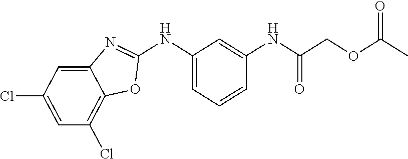 [SMILES: CC(=O)OCC(=O)Nc1cccc(Nc2nc3cc(Cl)cc(Cl)c3o2)c1]

The title compound was obtained in a similar manner to Example 4-1, except that N¹-(5,7-dichlorobenzo[d]oxazol-2-yl)benzene-1,3-diamine prepared according to Example 2-12 was used instead of N¹-(5-fluorobenzo[d]oxazol-2-yl)benzene-1,4-diamine.
Pale brown solid (75.6%);
m.p. 95˜97° C.;

¹H NMR (400 MHz, Acetone-d₆) δ 9.89 (s, 1H), 9.32 (s, 1H), 8.10 (t, J=1.6 Hz, 1H), 7.66 (dq, J=8.0, 1.1 Hz, 1H), 7.42-7.39 (m, 2H), 7.35 (t, J=7.8 Hz, 1H), 7.22 (d, J=1.6 Hz, 1H), 4.70 (s, 2H), 2.14 (s, 3H);

HR-FABMS Calcd for C₁₇H₁₃Cl₂N₃O₄(M+H)⁺: 394.0361, Found: 394.0361.

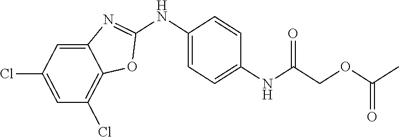 [SMILES: CC(=O)OCC(=O)Nc1ccc(Nc2nc3cc(Cl)cc(Cl)c3o2)cc1]

The title compound was obtained in a similar manner to Example 4-1, except that N¹-(5,7-dichlorobenzo[d]oxazol-2-yl)benzene-1,4-diamine prepared according to Example 2-13 was used instead of N¹-(5-fluorobenzo[d]oxazol-2-yl)benzene-1,4-diamine.
Pale brown solid (82.0%);
m.p. 262˜263° C.;

¹H NMR (400 MHz, DMSO-d₆) δ 11.06 (s, 1H), 10.05 (s, 1H), 7.65 (d, J=9.2 Hz, 2H), 7.58 (d, J=8.8 Hz, 2H), 7.50 (d, J=2.0 Hz, 1H), 7.33 (d, J=2.0 Hz, 1H), 4.63 (s, 2H), 2.12 (s, 3H);

HR-FABMS Calcd for C₁₇H₁₃Cl₂N₃O₄(M+H)⁺: 394.0361, Found: 394.0357.

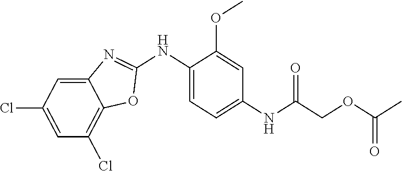 [SMILES: COc1cc(NC(=O)COC(C)=O)ccc1Nc1nc2cc(Cl)cc(Cl)c2o1]

The title compound was obtained in a similar manner to Example 4-1, except that N¹-(5,7-dichlorobenzo[d]oxazol-2-yl)-2-methoxybenzene-1,4-diamine prepared according to Example 2-14 was used instead of N¹-(5-fluorobenzo[d]oxazol-2-yl)benzene-1,4-diamine.
Greenish brown solid (73.7%);
m.p. 185˜187° C.;

¹H NMR (400 MHz, Acetone-d₆) δ 9.24 (s, 1H), 9.10 (s, 1H), 8.27 (d, J=8.8 Hz, 1H), 7.66 (d, J=2.4 Hz, 1H), 7.39 (d, J=2.0 Hz, 1H), 7.20 (d, J=2.0 Hz, 1H), 7.18 (dd, J=8.8, 2.4 Hz, 1H), 4.68 (s, 2H), 3.92 (s, 3H), 2.13 (s, 3H);

HR-FABMS Calcd for C₁₈H₁₅Cl₂N₃O₅ (M+H)⁺: 424.0462, Found: 424.0466.

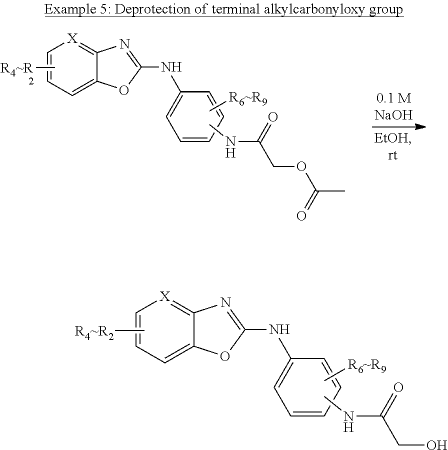

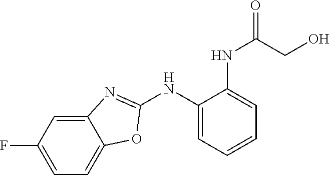 [SMILES: O=C(CO)Nc1ccccc1Nc1nc2cc(F)ccc2o1]

1 equivalent of 2-(2-(5-fluorobenzo[d]oxazol-2-ylamino)phenylamino)-2-oxoethyl acetate prepared according to Example 4-1 was dissolved in ethanol, 0.1 M NaOH aqueous solution was added thereto, and stirring was carried out at room temperature for 30 minutes. After a solvent was removed under reduced pressure, the reaction product was extracted with ethyl acetate and washed with saturated aqueous sodium chloride solution. This product was dried over anhydrous magnesium sulfate and filtered, and the solvent was removed under reduced pressure to obtain the title compound.
Pale brown solid (88.0%);
m.p. 174˜175° C.;

¹H NMR (400 MHz, Acetone-d₆) δ 9.51 (s, 1H), 9.23 (s, 1H), 7.95 (dd, J=7.8, 1.4 Hz, 1H), 7.73 (d, J=8.4 Hz, 1H), 7.33 (dd, J=8.4, 4.4 Hz, 1H), 7.30 (td, J=7.7, 1.7 Hz, 1H), 7.23 (td, J=7.7, 1.3 Hz, 1H), 7.11 (dd, J=9.2, 2.4 Hz, 1H), 6.871 (ddd, J=9.8, 8.8, 2.6 Hz, 1H), 4.919 (t, J=5.6 Hz, 1H), 4.175 (d, J=6.0 Hz, 2H);

HR-FABMS Calcd for C₁₅H₁₂FN₃O₃(M+H)⁺: 302.0935, Found: 302.0945.

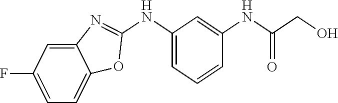 [SMILES: O=C(CO)Nc1cccc(Nc2nc3cc(F)ccc3o2)c1]

The title compound was obtained in a similar manner to Example 5-1, except that 2-(3-(5-fluorobenzo[d]oxazol-2-ylamino)phenylamino)-2-oxoethyl acetate prepared according to Example 4-2 was used instead of 2-(2-(5-fluorobenzo[d]oxazol-2-ylamino)phenylamino)-2-oxoethyl acetate.
Pale brown solid (93.0%);
m.p. 227˜228° C.;

¹H NMR (400 MHz, DMSO-d₆) δ 10.73 (s, 1H), 9.69 (s, 1H), 8.04 (t, J=1.6 Hz, 1H), 7.51 (d, J=7.6 Hz, 1H), 7.49 (dd, J=8.8, 4.4 Hz, 1H), 7.35 (d, J=8.0 Hz, 1H), 7.31-7.27 (m, 2H), 6.97-6.91 (m, 1H), 5.59 (t, J=6.0 Hz, 1H), 4.01 (d, J=6.0 Hz, 2H);

HR-FABMS Calcd for C₁₅H₁₂FN₃O₃(M+H)⁺: 302.0935, Found: 302.0942.

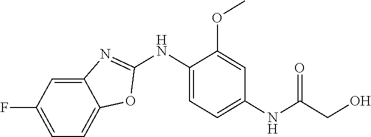 [SMILES: COc1cc(NC(=O)CO)ccc1Nc1nc2cc(F)ccc2o1]

The title compound was obtained in a similar manner to Example 5-1, except that 2-(4-(5-fluorobenzo[d]oxazol-2-ylamino)-3-methoxyphenylamino)-2-oxoethyl acetate prepared according to Example 4-3 was used instead of 2-(2-(5-fluorobenzo[d]oxazol-2-ylamino)phenylamino)-2-oxoethyl acetate.
Pale brown solid (42.6%);
m.p. 205˜206° C.;

¹H NMR (400 MHz, Acetone-d₆) δ 9.11 (s, 1H), 8.70 (s, 1H), 8.33 (d, J=8.8 Hz, 1H), 7.73 (d, J=2.4 Hz, 1H), 7.38-7.33 (m, 2H), 7.17 (dd, J=9.0, 2.6 Hz, 1H), 6.87 (ddd, J=9.8, 8.8, 2.6 Hz, 1H), 4.82 (t, J=5.6 Hz, 1H), 4.11 (d, J=5.6 Hz, 2H), 3.92 (s, 3H);

HR-FABMS Calcd for C₁₆H₁₄FN₃O₄ (M+H)⁺: 332.1046, Found: 332.1045.

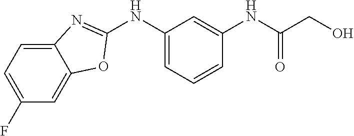 [SMILES: O=C(CO)Nc1cccc(Nc2nc3ccc(F)cc3o2)c1]

The title compound was obtained in a similar manner to Example 5-1, except that 2-(3-(6-fluorobenzo[d]oxazol-2-ylamino)phenylamino)-2-oxoethyl acetate prepared according to Example 4-4 was used instead of 2-(2-(5-fluorobenzo[d]oxazol-2-ylamino)phenylamino)-2-oxoethyl acetate.
Pale brown solid (100.0%);
m.p. 229˜230° C.;

¹H NMR (400 MHz, DMSO-d₆) δ 9.58 (s, 1H), 9.12 (s, 1H), 8.17 (t, J=2.0 Hz, 1H), 7.68 (ddd, J=8.2, 2.1, 0.9 Hz, 1H), 7.44-7.40 (m, 2H), 7.33 (t, J=8.2 Hz, 1H), 7.27 (dd, J=8.4, 2.4 Hz, 1H), 7.03 (ddd, J=10.0, 8.7, 2.6 Hz, 1H), 4.78 (t, J=5.6 Hz, 1H), 4.13 (d, J=5.6 Hz, 2H);

HR-FABMS Calcd for C₁₅H₁₂FN₃O₃(M+H)⁺: 302.0935, Found: 302.0943.

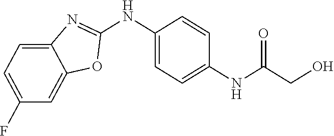 [SMILES: O=C(CO)Nc1ccc(Nc2nc3ccc(F)cc3o2)cc1]

The title compound was obtained in a similar manner to Example 5-1, except that 2-(4-(6-fluorobenzo[d]oxazol-2-ylamino)phenylamino)-2-oxoethyl acetate prepared according to Example 4-5 was used instead of 2-(2-(5-fluorobenzo[d]oxazol-2-ylamino)phenylamino)-2-oxoethyl acetate.
Pale brown solid (100%);
m.p. 209˜211° C.;

¹H NMR (400 MHz, Acetone-d₆) δ 9.49 (s, 1H), 9.08 (s, 1H), 7.79 (s, 4H), 7.40 (dd, J=8.8, 4.8 Hz, 1H), 7.26 (dd, J=8.4, 2.4 Hz, 1H), 7.02 (ddd, J=10.0, 8.6, 2.6 Hz, 1H), 4.76 (t, J=5.6 Hz, 1H), 4.09 (d, J=6.0 Hz, 2H);

HR-FABMS Calcd for C₁₅H₁₂FN₃O₃ (M+H)⁺: 302.0935, Found: 302.0942.

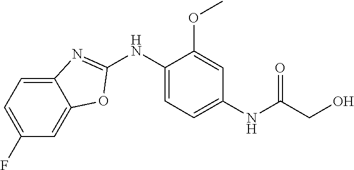

The title compound was obtained in a similar manner to Example 5-1, except that 2-(4-(6-fluorobenzo[d]oxazol-2-ylamino)-3-methoxyphenylamino)-2-oxoethyl acetate prepared according to Example 4-6 was used instead of 2-(2-(5-fluorobenzo[d]oxazol-2-ylamino)phenylamino)-2-oxoethyl acetate.
White solid (84.0%);
m.p. 205˜206° C.;

¹H NMR (400 MHz, Acetone-d₆) δ 9.10 (s, 1H), 8.70 (s, 1H), 8.36 (d, J=8.8 Hz, 1H), 7.72 (d, J=2.0 Hz, 1H), 7.39 (dd, J=8.4, 4.8 Hz, 1H), 7.34 (dd, J=8.6, 2.2 Hz, 1H), 7.26 (dd, J=8.4, 2.4 Hz, 1H), 7.02 (ddd, J=10.0, 8.6, 2.6 Hz, 1H), 4.81 (t, J=5.6 Hz, 1H), 4.11 (d, J=5.6 Hz, 2H), 3.92 (s, 3H);

HR-FABMS Calcd for C₁₆H₁₄FN₃O₄(M+H)⁺: 332.1046, Found: 332.1048.

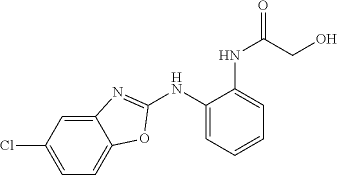 [SMILES: O=C(CO)Nc1ccccc1Nc1nc2cc(Cl)ccc2o1]

The title compound was obtained in a similar manner to Example 5-1, except that 2-(2-(5-chlorobenzo[d]oxazol-2-ylamino)phenylamino)-2-oxoethyl acetate prepared according to Example 4-7 was used instead of 2-(2-(5-fluorobenzo[d]oxazol-2-ylamino)phenylamino)-2-oxoethyl acetate.
Pale brown solid (81.0%);
m.p. 173˜174° C.;

¹H NMR (400 MHz, Acetone-d₆) δ 9.51 (s, 1H), 9.31 (s, 1H), 7.94 (dd, J=7.8, 1.4 Hz, 1H), 7.73 (dt, J=8.0, 2.1 Hz, 1H), 7.39 (d, J=8.4 Hz, 1H), 7.36 (d, J=2.4 Hz, 1H), 7.30 (td, J=7.7, 1.7 Hz, 1H), 7.24 (td, J=7.6, 1.6 Hz, 1H), 7.12 (dd, J=8.6, 2.2 Hz, 1H), 4.92 (t, J=5.7 Hz, 1H), 4.13 (d, J=6.0 Hz, 2H);

HR-FABMS Calcd for C₁₅H₁₂CIN₃O₃(M+H)⁺: 318.0640, Found: 318.0650.

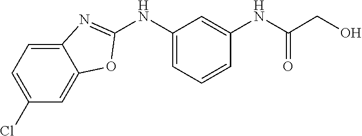 [SMILES: O=C(CO)Nc1cccc(Nc2nc3ccc(Cl)cc3o2)c1]

The title compound was obtained in a similar manner to Example 5-1, except that 2-(3-(6-chlorobenzo[d]oxazol-2-ylamino)phenylamino)-2-oxoethyl acetate prepared according to Example 4-9 was used instead of 2-(2-(5-fluorobenzo[d]oxazol-2-ylamino)phenylamino)-2-oxoethyl acetate.
Pale brown solid (88.2%);
m.p. 235˜237° C.;

¹H NMR (400 MHz, DMSO-d₆) δ 10.75 (s, 1H), 9.69 (s, 1H), 8.05 (t, J=2.0 Hz, 1H), 7.68 (d, J=2.0 Hz, 1H), 7.51 (dq, J=8.0, 1.2 Hz, 1H), 7.43 (d, J=8.4 Hz, 1H), 7.34 (dt, J=8.8, 1.6 Hz, 1H), 7.30 (d, J=8.0 Hz, 1H), 7.26 (dd, J=8.4, 2.0 Hz, 1H), 5.58 (t, J=5.8 Hz, 1H), 4.01 (d, J=5.6 Hz, 2H);

HR-FABMS Calcd for C₁₅H₁₂CIN₃O₃ (M+H)⁺: 318.0640, Found: 318.0650.

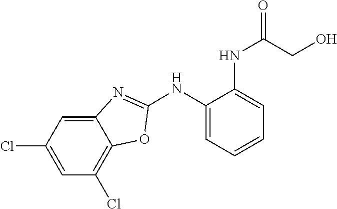 [SMILES: O=C(CO)Nc1ccccc1Nc1nc2cc(Cl)cc(Cl)c2o1]

The title compound was obtained in a similar manner to Example 5-1, except that 2-(2-(5,7-dichlorobenzo[d]oxazol-2-ylamino)phenylamino)-2-oxoethyl acetate prepared according to Example 4-10 was used instead of 2-(2-(5-fluorobenzo[d]oxazol-2-ylamino)phenylamino)-2-oxoethyl acetate.
White solid (31.2%);
m.p. 147˜148° C.;

¹H NMR (400 MHz, Acetone-d₆) δ 9.49 (s, 2H), 7.96 (dd, J=8.0, 1.6 Hz, 1H), 7.75 (dt, J=8.0, 2.4 Hz, 1H), 7.33 (d, J=2.0 Hz, 1H), 7.31 (dd, J=7.8, 1.8 Hz, 1H), 7.26 (td, J=7.7, 1.7 Hz, 1H), 7.20 (d, J=1.6 Hz, 1H), 4.92 (s, 1H), 4.17 (d, J=5.6 Hz, 2H);

HR-FABMS Calcd for C₁₅H₁₁Cl₂N₃O₃ (M+H)⁺: 352.0250, Found: 352.0255.

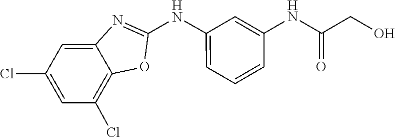 [SMILES: O=C(CO)Nc1cccc(Nc2nc3cc(Cl)cc(Cl)c3o2)c1]

The title compound was obtained in a similar manner to Example 5-1, except that 2-(3-(5,7-dichlorobenzo[d]oxazol-2-ylamino)phenylamino)-2-oxoethyl acetate prepared according to Example 4-11 was used instead of 2-(2-(5-fluorobenzo[d]oxazol-2-ylamino)phenylamino)-2-oxoethyl acetate.
Pale brown solid (100.0%);
m.p. 269˜270° C.;

¹H NMR (400 MHz, Acetone-d₆) δ 9.91 (s, 1H), 9.16 (s, 1H), 8.20 (s, 1H), 7.68 (dq, J=8.0, 1.1 Hz, 1H), 7.48 (dd, J=8.4, 1.2 Hz, 1H), 7.41 (d, J=2.0 Hz, 1H), 7.37 (t, J=8.2 Hz, 1H), 7.22 (d, J=1.6 Hz, 1H), 4.79 (s, 1H), 4.12 (s, 2H);

HR-FABMS Calcd for C₁₅H₁₁Cl₂N₃O₃(M+H)⁺: 352.0250, Found: 352.0258.

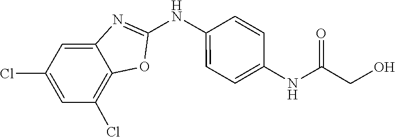 [SMILES: O=C(CO)Nc1ccc(Nc2nc3cc(Cl)cc(Cl)c3o2)cc1]

The title compound was obtained in a similar manner to Example 5-1, except that 2-(4-(5,7-dichlorobenzo[d]oxazol-2-ylamino)phenylamino)-2-oxoethyl acetate prepared according to Example 4-12 was used instead of 2-(2-(5-fluorobenzo[d]oxazol-2-ylamino)phenylamino)-2-oxoethyl acetate.
Pale brown solid (100.0%);
m.p. 209˜211° C.;

¹H NMR (400 MHz, DMSO-d₆) δ 11.03 (s, 1H), 9.63 (s, 1H), 7.70 (d, J=8.8 Hz, 2H), 7.63 (d, J=9.2 Hz, 2H), 7.49 (d, J=2.0 Hz, 1H), 7.33 (d, J=2.0 Hz, 1H), 5.64 (t, J=6.0 Hz, 1H), 3.98 (d, J=6.0 Hz, 2H);

HR-FABMS Calcd for C₁₅H₁₁Cl₂N₃O₃(M+H)⁺: 352.0250, Found: 352.0259.

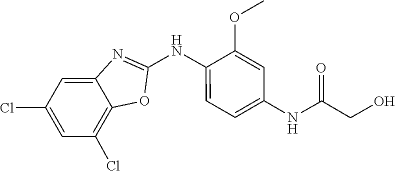 [SMILES: COc1cc(NC(=O)CO)ccc1Nc1nc2cc(Cl)cc(Cl)c2o1]

The title compound was obtained in a similar manner to Example 5-1, except that 2-(4-(5,7-dichlorobenzo[d]oxazol-2-ylamino)-3-methoxyphenylamino)-2-oxoethyl acetate prepared according to Example 4-13 was used instead of 2-(2-(5-fluorobenzo[d]oxazol-2-ylamino)phenylamino)-2-oxoethyl acetate.
Yellow solid (68.2%);
m.p. 201˜202° C.;

¹H NMR (400 MHz, Acetone-d₆) δ 9.14 (s, 1H), 9.08 (s, 1H), 8.27 (d, J=8.4 Hz, 1H), 7.74 (d, J=2.0 Hz, 1H), 7.38 (d, J=1.6 Hz, 1H), 7.36 (dd, J=8.8, 2.4 Hz, 1H), 7.19 (d, J=2.0 Hz, 1H), 4.84 (s, 1H), 4.11 (s, 2H), 3.93 (s, 3H);

HR-FABMS Calcd for C₁₆H₁₃Cl₂N₃O₄ (M+H)⁺: 382.0361, Found: 382.0357.

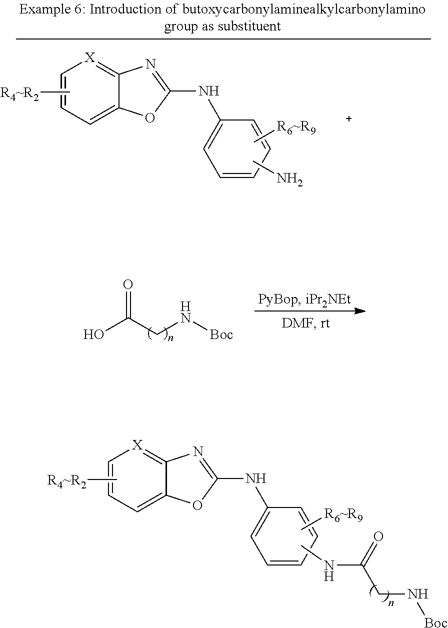

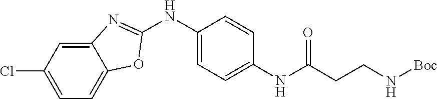 [SMILES: CC(C)(C)OC(=O)NCCC(=O)Nc1ccc(Nc2nc3cc(Cl)ccc3o2)cc1]

1 equivalent of N¹-(5-chlorobenzo[d]oxazol-2-yl)benzene-1,4-diamine prepared according to Example 2-8 was dissolved in dimethylformamide, (benzotriazol-1-yloxy)tripyrrolidinophosphonium hexafluorophosphate, 3-(tert-butoxycarbonylamino)propanoic add, and diisopropylethylamine were added thereto in an amount of each 1 equivalent, and stirring was carried out at room temperature for 16 hours. After 10% aqueous hydrochloric add solution was added to the reaction product and this product was extracted with ethyl acetate, the resulting product was washed with saturated aqueous sodium bicarbonate solution and saturated aqueous sodium chloride solution. The obtained organic layer was dried over anhydrous magnesium sulfate and filtered, and then the solvent was concentrated under reduced pressure to obtain the title compound.
Brown solid (56.0%);
m.p. 207˜208° C.;

¹H NMR (400 MHz, Acetone-d₆) δ 7.97 (s, 1H), 7.77 (d, J=9.2 Hz, 2H), 7.70 (d, J=9.2 Hz, 2H), 7.42 (d, J=2.0 Hz, 1H), 7.38 (d, J=8.4 Hz, 1H), 7.12 (dd, J=8.4 Hz, 1H), 6.03 (brs, 1H), 3.41 (q, J=6.4 Hz, 2H), 2.59 (t, J=6.6 Hz, 2H), 1.41 (s, 9H), 1.32 (s, 9H);

HR-FABMS Calcd for C₂₁H₂₄CIN₄O₄(M+H)⁺: 431.1486, Found: 431.1484.

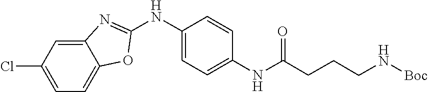 [SMILES: CC(C)(C)OC(=O)NCCCC(=O)Nc1ccc(Nc2nc3cc(Cl)ccc3o2)cc1]

The title compound was obtained in a similar manner to Example 6-1, except that 4-(tert-butoxycarbonylamino)butanoic acid was used instead of 3-(tert-butoxycarbonylamino)propanoicadd.
White solid (20.0%);
m.p. 203˜206° C.;

¹H NMR (400 MHz, Acetone-d₆) δ 9.57 (s, 1H), 9.23 (s, 1H), 7.76 (d, J=9.2 Hz, 2H), 7.70 (d, J=8.0 Hz, 2H), 7.42 (d, J=2.4 Hz, 1H), 7.38 (d, J=8.4 Hz, 1H), 7.12 (dd, J=8.4 Hz, 1H), 6.06 (brs, 1H), 3.16 (q, J=6.5 Hz, 2H), 2.40 (t, J=7.4 Hz, 2H), 1.86 (m, 2H), 1.41 (s, 9H);

HR-FABMS Calcd for C₂₂H₂₆CIN₄O₄(M+H)⁺: 445.1643, Found: 445.1639.

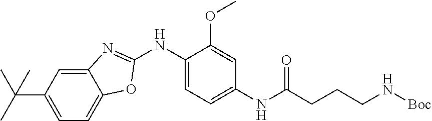 [SMILES: COc1cc(NC(=O)CCCNC(=O)OC(C)(C)C)ccc1Nc1nc2cc(C(C)(C)C)ccc2o1]

The title compound was obtained in a similar manner to Example 6-1, except that N¹-(5-tert-butylbenzo[d]oxazol-2-yl)-2-methoxybenzene-1,4-diamine was used instead of N¹-(5-chlorobenzo[d]oxazol-2-yl)benzene-1,4-diamine, and 4-(tert-butoxycarbonylamino)butanoic add was used instead of 3-(tert-butoxycarbonylamino)propanoic add.
Brown oil (60.2%);

¹H NMR (400 MHz, DMSO-d₆) δ 10.43 (s, 1H), 9.82 (s, 1H), 7.65 (d, J=9.2 Hz, 2H), 7.56 (d, J=8.8 Hz, 2H), 7.45 (s, 1H), 7.44 (d, J=8.4 Hz, 1H), 7.12 (d, J=6.8 Hz, 1H), 6.83 (s, 1H), 2.96 (d, J=6.4 Hz, 2H), 2.27 (t, J=6.8 Hz, 2H), 1.68 (d, J=7.2 Hz, 2H), 1.38 (s, 9H), 1.32 (s, 9H);

HR-FABMS Calcd for C₂₆H₃₅N₄O₄(M+H)⁺: 467.2653, Found: 467.2656.

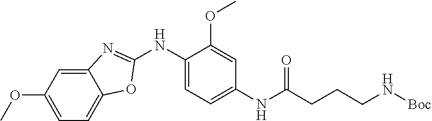 [SMILES: COc1ccc2oc(Nc3ccc(NC(=O)CCCNC(=O)OC(C)(C)C)cc3OC)nc2c1]

The title compound was obtained in a similar manner to Example 6-1, except that N¹-(5-methoxybenzo[d]oxazol-2-yl)-2-methoxybenzene-1,4-diamine was used instead of N¹-(5-chlorobenzo[d]oxazol-2-yl)benzene-1,4-diamine, and 4-(tert-butoxycarbonylamino)butanoic add was used instead of 3-(tert-butoxycarbonylamino)propanoic add.
Ivory solid (90.0%);
m.p. 190˜192° C.;

¹H NMR (400 MHz, DMSO-d₆) δ 10.45 (s, 1H), 9.82 (s, 1H), 7.63 (d, J=8.8 Hz, 2H), 7.56 (d, J=9.2 Hz, 2H), 7.34 (d, J=9.2 Hz, 1H), 7.02 (d, J=2.4 Hz, 1H), 6.83 (t, J=4.8 Hz, 1H), 6.66 (dd, J=8.4, 2.8 Hz, 1H), 3.77 (s, 3H), 2.96 (dd, J=13.2, 6.8 Hz, 2H), 2.27 (t, J=7.6 Hz, 2H), 1.69 (t, J=7.2 Hz, 2H), 1.38 (s, 9H);

HR-FABMS Calcd for C₂₃H₂₉N₄O₅ (M+H)⁺: 441.2132, Found: 441.2128.

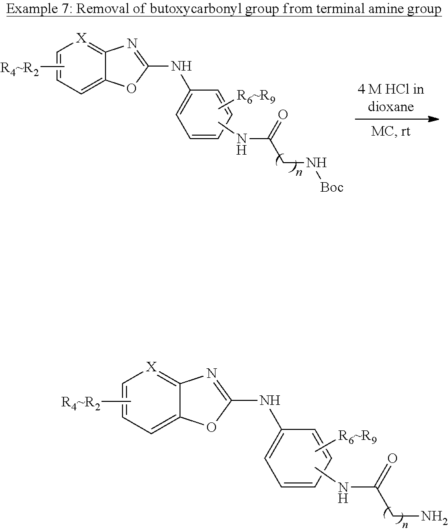

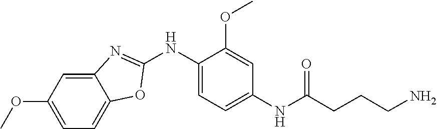 [SMILES: COc1ccc2oc(Nc3ccc(NC(=O)CCCN)cc3OC)nc2c1]

1 equivalent of tert-Butyl 4-(3-methoxy-4-(5-methoxybenzo[d]oxazol-2-ylamino)phenylamino)-4-oxobutylcarbamate prepared according to Example 6-4 was dissolved in chloroform, a 4M hydrochloric acid-dioxane solution were added thereto, and then stirring was carried out at room temperature for 3 hours. Then, the solvent was removed by evaporation under reduced pressure to obtain the title compound.
Ivory solid (84.7%);
m.p. 216˜219° C.;

¹H NMR (400 MHz, DMSO-d₆) δ10.51 (s, 1H), 10.01 (s, 1H), 7.86 (s, 2H), 7.65 (d, J=9.2 Hz, 2H), 7.58 (d, J=8.8 Hz, 2H), 7.35 (d, J=8.8 Hz, 1H), 7.01 (d, J=2.0 Hz, 1H), 6.67 (dd, J=8.4, 2.8 Hz, 1H), 3.77 (s, 3H), 2.87-2.80 (m, 2H), 2.42 (t, J=7.2 Hz, 2H), 1.86 (t, J=8.0 Hz, 2H);

HR-FABMS Calcd for C₁₈H₂₁N₄O₃(M+H)⁺: 341.1608, Found: 325.1607.

### Experimental Example 1: LTB4 Inhibition Effect

In order to confirm the effect on BLT2, PANC-1 cells, a human pancreatic cancer cell line, were treated with LY255283 (50 μM), a known BLT2 antagonist or the compound prepared according to each of Examples (10 μM) for 2 hours, and were then treated with compound A (100 nM), a BLT2 agonist. Further, in order to confirm the effect on BLT1 receptors, PANC-1 cells were pretreated with the compound prepared according to each of Examples, treated with LTB4 (100 nM), and then cultured for 48 hours.

In order to measure the migration of cells, experiments were performed using a 96-well disposable transwell unit (96-well ChemoTx Disposable Chemotaxis System transwell unit, Neuroprobe Inc., USA) including a polycarbonate membrane having 8 μm pores. Before experiments, the day before, a process of coating the bottom portion of the transwell unit (bottom well of filter) with 10 μL of fibronectin (10 μg/mL, Santa Cruz, USA) as a chemoattractant and drying it at room temperature was performed. Thereafter, cells were prepared in a high glucose DMEM medium (high glucose Dulbecco's Modified Eagle's Medium, Welgene Inc., South Korea) under a condition of no FBS at a density of 5×10⁴ cells per well, 25 μL of cells were dispensed in the upper portion of the transwell unit (upper well of filter). 30 μL of DMEM medium containing 3% FBS as a chemical attractant along with fibronectin was introduced to a bottom plate and cells were cultured for 16 hours. After being cultured, the cells were dyed using a Diff-quick (Kobe, Japan) reagent, and then pictures of five or more randomly selected locations were taken at 200× magnification, the cells were counted, and cell migration was analyzed.

Moreover, in order to measure the invasion of cells, experiments were performed using a 24-well transwell unit (Coming, USA) including a polycarbonate membrane having 8 μm pores. Before experiments, the day before, a process of coating the inner portion of a transwell insert with 10 μL of Matrigel (0.5 mg/mL, BD Bioscience, USA) and drying it at room temperature was performed. Thereafter, cells were prepared in the DMEM medium under a condition of no FBS at a density of 2×10⁶ cells per well, and cells were dispensed in the transwell insert. 500 μL of DMEM medium containing 10% FBS was introduced to a bottom plate and cultured for 24 hours. After being cultured, the cells were fixed with cold methanol for 10 minutes, dyed with hematoxylin for 10 minutes and with eosin for 1 minute, and then pictures of five or more randomly selected locations were taken at 200× magnification, the cells were counted, and the degree of cell invasion was analyzed.

### Experimental Example 2: Search for BLT1 and/or BLT2 Inhibitors

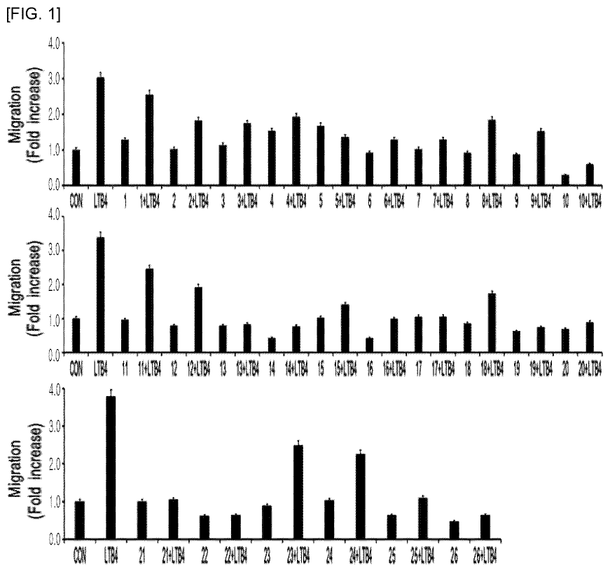

Compounds that inhibit the migration of cells increased by treating LTB4 were searched, and the results thereof are shown in FIG. 1. As shown in FIG. 1, inhibitory activity was observed in assays 5, 6, 7, 10, 13, 14, 15, 17, 18, 20, 21, 22, and 26.

### Experimental Example 3: Search for BLT2 Inhibitory Substances

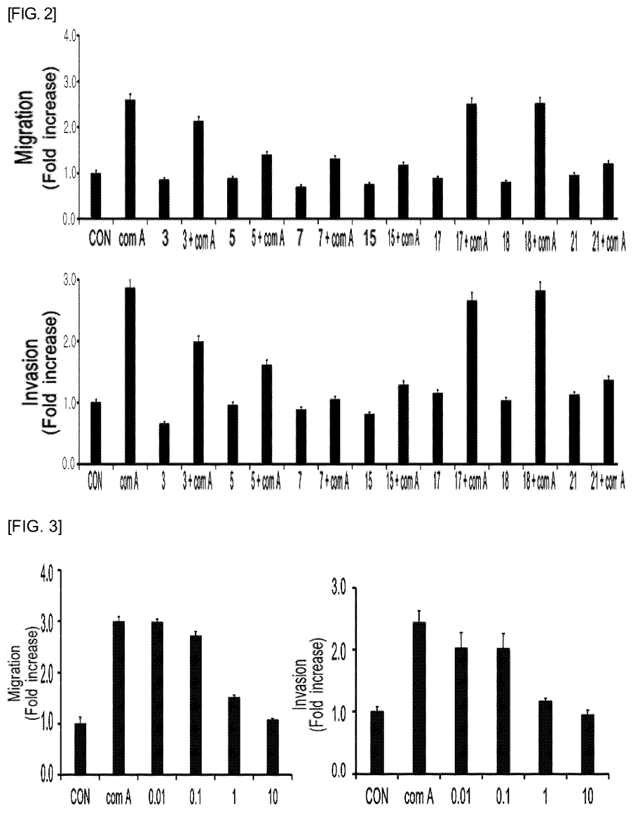

It was confirmed whether the substances (Assays 1 to 30) synthesized according to Examples inhibit the migration of PANC-1 cells increased when treated with Compound A, a BLT2 agonist, and the results thereof are shown in FIG. 2. As shown in FIG. 2, it was confirmed that the migration and invasion of cells increased by Compound A, a BLT2 agonist, were significantly inhibited by the compounds of Assays 5, 7, 15, and 21.

Overall, among Assays 5, 6, 7, 10, 13, 14, 15, 17, 18, 20, 21, 22, and 26, which are substances that inhibit the migration and invasion of cells identified in Experimental Example 2, four compounds, Assays 5, 7, 15, and 21, were confirmed to inhibit the migration and invasion of cells via BLT2.

### Experimental Example 4: Dose-Dependent Inhibition of Cell Migration by BLT2

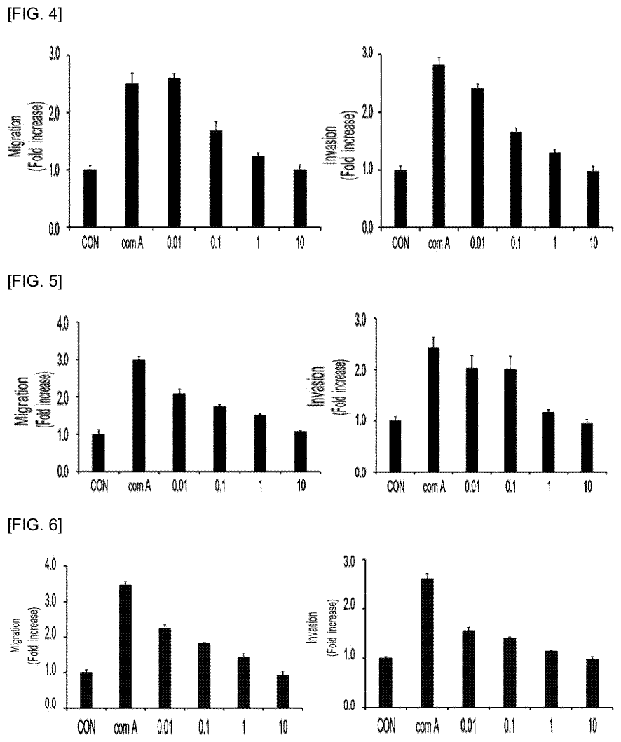

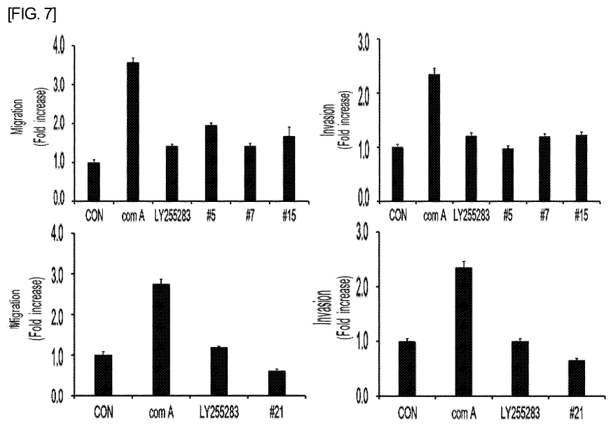

The compounds of Assays 5, 7, 15, and 21, which were confirmed to inhibit an increase in the migration and invasion of cells caused by Compound A, a BLT2 agonist in Experimental Example 3, were treated at various concentrations (0.01 to 10 μM) to determine concentration-dependent effects on cell migration and invasion, and the results thereof are shown in FIGS. 3 to 6, respectively. As shown in FIGS. 3 to 6, it was confirmed that these compounds reduce an increase in the migration and invasion of cells caused by Compound A in a concentration-dependent manner.

### Experimental Example 5: Comparison with BLT2 Receptor Antagonists

For comparison with the inhibitory effect of LY255283, a known BLT2 receptor antagonist, on an increase in the migration and invasion of cells caused by Compound A, migration and invasion were confirmed in cells treated with the compounds of Assay 5, 7, 15, and 21 used in Experimental Example 4 and LY255283 at a concentration of 10 μM, respectively, and the results thereof are shown in FIG. 7. As shown in FIG. 7, all of the compounds were confirmed to have the same inhibitory ability as LY255283 or similar inhibitory ability to LY255283 on an increase in the migration and invasion of cells by Compound A.

It will be appreciated by those skilled in the art that the present invention as described above may be implemented into other specific forms without departing from the technical spirit thereof or essential characteristics. Thus, it is to be appreciated that embodiments described above are intended to be illustrative in all aspects, and not restrictive. The scope of the present invention is represented by the appended claims to be described below rather than the detailed description, and it is to be interpreted that the meaning and scope of the appended claims and all the changes or modified forms derived from equivalents thereof come within the scope of the present invention.

1. A compound represented by Formula 1 or a pharmaceutically acceptable salt thereof: 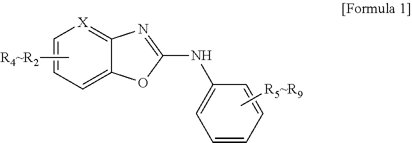
in Formula 1,
X is N or CR₁,
R₁ to R₄ are each independently hydrogen, halogen, nitro, C₁₋₄ alkyl, or C₁₋₄ alkoxy, and
R₅ to R₉ are each independently hydrogen, amino, nitro, C₁₋₄ alkoxy, hydroxy-C₁₋₄ alkylcarbonylamino, C₁₋₄ alkylcarbonyloxy-C₁₋₄ alkylcarbonylamino, amino-C₁₋₄ alkylcarbonylamino, or tert-butoxycarbonylamino-C₁₋₄ alkylcarbonylamino.

2. The compound or pharmaceutically acceptable salt thereof of claim 1,
wherein R₁ to R₄ are each independently hydrogen, fluoro, chloro, bromo, nitro, ethyl, tert-butyl, or methoxy.

3. The compound or pharmaceutically acceptable salt thereof of claim 1,
wherein R₅ to R₉ are each independently hydrogen, amino, nitro, methoxy, hydroxymethylcarbonylamino, methylcarbonyloxymethylcarbonylamino, tert-butoxycarbonylaminopropylcarbonylamino. tert-butoxycarbonylaminoethylcarbonylamino, or aminopropylcarbonylamino.

4. The compound or pharmaceutically acceptable salt thereof of claim 1,
wherein the compound is as follows:
1. 5-fluoro-N-(2-nitrophenyl)benzo[d]oxazol-2-amine,
2. 5-fluoro-N-(3-nitrophenyl)benzo[d]oxazol-2-amine,
3. 5-fluoro-N-(2-methoxy-4-nitrophenyl)benzo[d]oxazol-2-amine,
4. 6-fluoro-N-(3-nitrophenyl)benzo[d]oxazol-2-amine,
5. 6-fluoro-N-(4-nitrophenyl)benzo[d]oxazol-2-amine,
6. 6-fluoro-N-(2-methoxy-4-nitrophenyl)benzo[d]oxazol-2-amine,
7. 5-chloro-N-(2-nitrophenyl)benzo[d]oxazol-2-amine,
8. 5-chloro-N-(4-nitrophenyl)benzo[d]oxazol-2-amine,
9. 6-chloro-N-(3-nitrophenyl)benzo[d]oxazol-2-amine,
10. 6-chloro-N-(2-methoxy-4-nitrophenyl)benzo[d]oxazol-2-amine,
11. 5,7-dichloro-N-(2-nitrophenyl)benzo[d]oxazol-2-amine,
12. 5,7-dichloro-N-(3-nitrophenyl)benzo[d]oxazol-2-amine,
13. 5,7-dichloro-N-(4-nitrophenyl)benzo[d]oxazol-2-amine,
14. 5,7-dichloro-N-(2-methoxy-4-nitrophenyl)benzo[d]oxazol-2-amine,
15. 5-chloro-N-(2-methoxy-4-nitrophenyl)-6-nitrobenzo[d]oxazol-2-amine,
16. 7-chloro-N-(2-methoxy-4-nitrophenyl)-5-nitrobenzo[d]oxazol-2-amine,
17. 5,7-dichloro-6-ethyl-N-(2-methoxy-4-nitrophenyl)benzo[d]oxazol-2-amine,
18. 5-tert-butyl-N-(2-methoxy-4-nitrophenyl)benzo[d]oxazol-2-amine,
19. 5-methoxy-N-(2-methoxy-4-nitrophenyl)benzo[d]oxazol-2-amine,
20. 6-bromo-N-(2-methoxy-4-nitrophenyl)oxazolo[4,5-b]pyridin-2-amine,
21. N1-(5-fluorobenzo[d]oxazol-2-yl)benzene-1,2-diamine,
22. N1-(5-fluorobenzo[d]oxazol-2-yl)benzene-1,3-diamine,
23. N1-(5-fluorobenzo[d]oxazol-2-yl)-2-methoxybenzene-1,4-diamine,
24. N1-(6-fluorobenzo[d]oxazol-2-yl)benzene-1,3-diamine,
25. N1-(6-fluorobenzo[d]oxazol-2-yl)benzene-1,4-diamine,
26. N1-(6-fluorobenzo[d]oxazol-2-yl)-2-methoxybenzene-1,4-diamine,
27. N1-(5-chlorobenzo[d]oxazol-2-yl)benzene-1,2-diamine,
28. N1-(5-chlorobenzo[d]oxazol-2-yl)benzene-1,4-diamine,
29. N1-(6-chlorobenzo[d]oxazol-2-yl)benzene-1,3-diamine,
30. N1-(6-chlorobenzo[d]oxazol-2-yl)-2-methoxybenzene-1,4-diamine,
31. N1-(5,7-dichlorobenzo[d]oxazol-2-yl)benzene-1,2-diamine,
32. N1-(5,7-dichlorobenzo[d]oxazol-2-yl)benzene-1,3-diamine,
33. N1-(5,7-dichlorobenzo[d]oxazol-2-yl)benzene-1,4-diamine,
34. N1-(5,7-dichlorobenzo[d]oxazol-2-yl)-2-methoxybenzene-1,4-diamine,
35. N1-(5-tert-butylbenzo[d]oxazol-2-yl)-2-methoxybenzene-1,4-diamine,
36. 2-methoxy-N1-(5-methoxybenzo[d]oxazol-2-yl)benzene-1,4-diamine,
37. 2-(6-chlorobenzo[d]oxazol-2-ylamino)-5-nitrophenol,
38. 2-(2-(5-fluorobenzo[d]oxazol-2-ylamino)phenylamino)-2-oxoethyl acetate,
39. 2-(3-(5-fluorobenzo[d]oxazol-2-ylamino)phenylamino)-2-oxoethyl acetate,
40. 2-(4-(5-fluorobenzo[d]oxazol-2-ylamino)-3-methoxyphenylamino)-2-oxoethyl acetate,
41. 2-(3-(6-fluorobenzo[d]oxazol-2-ylamino)phenylamino)-2-oxoethyl acetate,
42. 2-(4-(6-fluorobenzo[d]oxazol-2-ylamino)phenylamino)-2-oxoethyl acetate,
43. 2-(4-(6-fluorobenzo[d]oxazol-2-ylamino)-3-methoxyphenylamino)-2-oxoethyl acetate,
44. 2-(2-(5-chlorobenzo[d]oxazol-2-ylamino)phenylamino)-2-oxoethyl acetate,
45. 2-(4-(6-chlorobenzo[d]oxazol-2-ylamino)phenylamino)-2-oxoethyl acetate,
46. 2-(3-(6-chlorobenzo[d]oxazol-2-ylamino)phenylamino)-2-oxoethyl acetate,
47. 2-(2-(5,7-dichlorobenzo[d]oxazol-2-ylamino)phenylamino)-2-oxoethyl acetate,
48. 2-(3-(5,7-dichlorobenzo[d]oxazol-2-ylamino)phenylamino)-2-oxoethyl acetate,
49. 2-(4-(5,7-dichlorobenzo[d]oxazol-2-ylamino)phenylamino)-2-oxoethyl acetate,
50. 2-(4-(5,7-dichlorobenzo[d]oxazol-2-ylamino)-3-methoxyphenylamino)-2-oxoethyl acetate,
51. N-(2-(5-fluorobenzo[d]oxazol-2-ylamino)phenyl)-2-hydroxyacetamide,
52. N-(3-(5-fluorobenzo[d]oxazol-2-ylamino)phenyl)-2-hydroxyacetamide,
53. N-(4-(5-fluorobenzo[d]oxazol-2-ylamino)-3-methoxyphenyl)-2-hydroxyacetamide,
54. N-(3-(6-fluorobenzo[d]oxazol-2-ylamino)phenyl)-2-hydroxyacetamide,
55. N-(4-(6-fluorobenzo[d]oxazol-2-ylamino)phenyl)-2-hydroxyacetamide,
56. N-(4-(6-fluorobenzo[d]oxazol-2-ylamino)-3-methoxyphenyl)-2-hydroxyacetamide,
57. N-(2-(5-chlorobenzo[d]oxazol-2-ylamino)phenyl)-2-hydroxyacetamide,
58. N-(3-(6-chlorobenzo[d]oxazol-2-ylamino)phenyl)-2-hydroxyacetamide,
59. N-(2-(5,7-dichlorobenzo[d]oxazol-2-ylamino)phenyl)-2-hydroxyacetamide,
60. N-(3-(5,7-dichlorobenzo[d]oxazol-2-ylamino)phenyl)-2-hydroxyacetamide,
61. N-(4-(5,7-dichlorobenzo[d]oxazol-2-ylamino)phenyl)-2-hydroxyacetamide,
62. N-(4-(5,7-dichlorobenzo[d]oxazol-2-ylamino)-3-methoxyphenyl)-2-hydroxyacetamide,
63. tert-butyl 3-(4-(5-chlorobenzo[d]oxazol-2-ylamino)phenylamino)-3-oxopropylcarbamate,
64. tert-butyl 4-(4-(5-chlorobenzo[d]oxazol-2-ylamino)phenylamino)-4-oxobutylcarbamate,
65. tert-butyl 4-(4-(5-tert-butylbenzo[d]oxazol-2-ylamino)-3-methoxyphenylamino)-4-oxobutylcarbamate,
66. tert-butyl 4-(3-methoxy-4-(5-methoxybenzo[d]oxazol-2-ylamino)phenylamino)-4-oxobutylcarbamate, or
67. 4-amino-N-(3-methoxy-4-(5-methoxybenzo[d]oxazol-2-ylamino)phenyl)butanamide.

5. A method of producing a compound represented by Formula 1 or a pharmaceutically acceptable salt thereof, the method comprising a 1 step of preparing a compound represented by Formula 3 by a cydization reaction of a hydroxyphenyl-thiourea derivative compound represented by Formula 2: 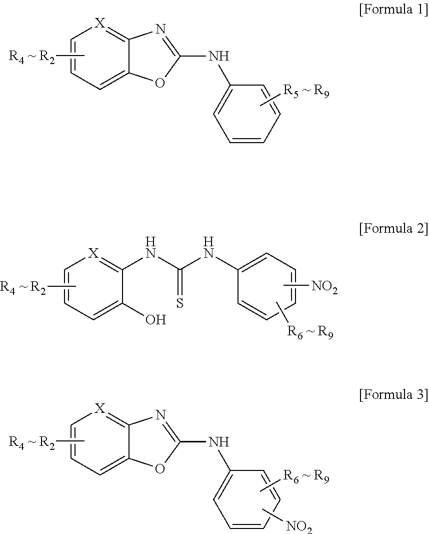
in Formula 1,
X is N or CR₁,
R₁ to R₄ are each independently hydrogen, halogen, nitro, C₁₋₄ alkyl, or C₁₋₄ alkoxy, and
R₅ to R₉ are each independently hydrogen, amino, nitro, C₁₋₄ alkoxy, hydroxy-C₁₋₄ alkylcarbonylamino, C₁₋₄ alkylcarbonyloxy-C₁₋₄ alkylcarbonylamino, amino-C₁₋₄ alkylcarbonylamino, or tert-butoxycarbonylamino-C₁₋₄ alkylcarbonylamino.

6. The method of claim 5,
wherein the hydroxyphenyl-thiourea derivative compound represented by Formula 2 is prepared by a 1′ step of reacting a 2-aminopyridin-3-ol or 2-aminophenol derivative compound represented by Formula 4 and a nitrophenyl isothiocyanate derivative compound represented by Formula 5:

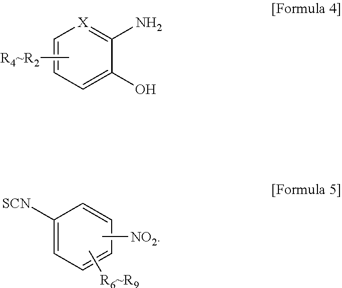

7. The method of claim 5, further comprising: a 2 step of reducing the product obtained from the 1 step to convert a nitro group substituted on a phenyl group into an amine group.

8. The method of claim 5,
wherein, when R₆ is alkoxy, the method further comprises a 1-1 step of converting the alkoxy group into a hydroxy group through a dealkylation reaction of the product obtained in the 1 step.

9. The method of claim 7, further comprising: a 3-1 step of reacting the product obtained in the 2 step with 2-halo-2-oxy-C₁₋₄ alkyl C₁₋₄ alkanoate to convert the amine group substituted on the phenyl ring into a C₁₋₄ alkylcarbonyloxy-C₁₋₄ alkylcarbonylamino group.

10. The method of claim 9, further comprising: a 4-1 step of reacting the product obtained in the 3-1 step under a base to convert the C₁₋₄ alkylcarbonyloxy-C₁₋₄ alkylcarbonylamino group substituted on the phenyl ring into a hydroxy-C₁₋₄ alkylcarbonylamino group.

11. The method of claim 7, further comprising: a 3-2 step of reacting the product obtained from the 2 step with tert-butoxycarbonylamino-C₁₋₄ alkanoic add to convert the amine group substituted on the phenyl ring into a tert-butoxycarbonylamino-C₁₋₄ alkylcarbonylamino group.

12. The method of claim 11, further comprising: a 4-2 step of reacting the product obtained from the 3-2 step with an add in an organic solvent to remove a tert-butoxycarbonyl group.

13. A pharmaceutical composition for preventing or treating diseases related to LTB4 receptor activity, the composition comprising a compound of Formula 1 or a pharmaceutically acceptable salt thereof, as an active ingredient: 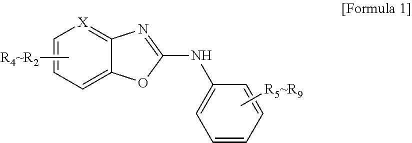
in Formula 1 above,
X is N or CR₁,
R₁ to R₄ are each independently hydrogen, halogen, nitro, C₁₋₄ alkyl, or C₁₋₄ alkoxy, and
R₅ to R₉ are each independently hydrogen, amino, nitro, C₁₋₄ alkoxy, hydroxy-C₁₋₄ alkylcarbonylamino, C₁₋₄ alkylcarbonyloxy-C₁₋₄ alkylcarbonylamino, amino-C₁₋₄ alkylcarbonylamino, or tert-butoxycarbonylamino-C₁₋₄ alkylcarbonylamino.

14. The pharmaceutical composition of claim 13,
wherein the pharmaceutical composition exhibits an effect of inhibiting an increase in cell migration, invasion or both due to activation of BLT2.

15. The pharmaceutical composition of claim 13,
wherein diseases related to LTB4 receptor activity are inflammatory diseases, cardiovascular diseases, or cancer diseases.
